(19) United States
(12) Patent Application Publication
(10) Pub. No.: US 2010/0056608 A1
(43) Pub. Date: Mar. 4, 2010

(54) METHODS FOR SCREENING AND TREATMENT INVOLVING THE GENES GYPC, AGPAT3, AGL, PVRL2, HMGB 3, HSDL2 AND/OR LDB2

(75) Inventors: Johan Bjorkegren, Stocksund (SE); Jesper Tegner, Stockholm (SE)

Correspondence Address: BROOKS KUSHMAN P.C., 1000 TOWN CENTER, TWENTY-SECOND FLOOR, SOUTHFIELD, MI, 48075, US

(73) Assignee: CLINICAL GENE NETWORKS AB, Stockholm (SE)

(21) Appl. No.: 12/515,344

(22) PCT Filed: Nov. 19, 2007
(86) PCT No.: PCT/SE07/50864

§ 371 Date: May 22, 2009

Related U.S. Application Data
(60) Provisional application No. 60/859,501, filed on Nov. 17, 2006.
(60) Provisional application No. 60/907,269, filed on Mar. 27, 2007.

(30) Foreign Application Priority Data
May 22, 2007 (SE) 0701219-8

(51) Int. Cl. A61K 31/7088; C12Q 1/68; C12N 5/10; A01K 67/00

(52) U.S. Cl. 514/44 A; 435/6; 435/325; 435/363; 435/366; 435/352; 800/13; 514/44 R

(57) ABSTRACT

The present invention relates to a method for identifying a compound as a candidate drug, comprising the steps a. bringing said compound into contact with a cell expressing the genes CYPC, AGPAT3, AGL, PVRL2, HMGB 3, HSDL2; and b. analyzing if said compound modulates the expression of at least one of said genes. It also relates to a method for identifying a compound as a candidate drug, comprising the steps a. bringing said compound into contact with a cell expressing the gene LDB2; and b. analyzing if said compound modulates the expression of LDB2. The invention further relates to genetically modified cells and animals useful in such methods and to methods for treatment of atherosclerosis, atherosclerosis-related diseases or inflammatory diseases, comprising the use of such identified compounds.

### FIELD OF THE INVENTION

The present invention relates to the field of drug development, and especially to screening for compounds that have therapeutic effect on atherosclerosis and atherosclerosis-related diseases and also other diseases involving inflammation and migration of leukocytes from the blood stream into the diseased tissue.

### BACKGROUND OF THE INVENTION

Despite improved lifestyles and effective lipid-lowering agents such as statins coronary artery disease (CAD) remains a leading health threat. CAD is a degenerative disease that develops over decades from the stress of circulating blood cells and other plasma constituents that gradually alters the artery wall composition (cellular and extracellular), eventually leading to the formation of atherosclerosis plaques. The rate of atherosclerosis development depends both on environmental pressures and on the genetic makeup of the individual. Environmental pressures relevant to CAD are mainly mediated by airborne pollutants (including cigarette smoke), infections, and food intake (calories and cholesterol) and by behavioral factors, in particular the degree of stress and exercise. The net effect of environmental pressures filtered through the individual genetic makeup is reflected by changes in blood flow and constituents. Over years, environmental and lifestyle factors alter gene expression in organs. Changes in the expression of genes related to energy metabolism and inflammation in the liver, fat, or skeletal muscle are believed to be particularly relevant for CAD. In turn, alterations in gene expression are reflected in the circulation, where metabolic and inflammatory markers synthesized in these organs can be detected. Thus, measurements of plasma constituents (e.g., cholesterol and triglycerides), blood glucose and insulin levels, and inflammatory markers such as C-reactive protein are the standard way to detect hypertriglyceridemia, hypercholesterolemia, insulin resistance, diabetes, states of inflammation and immune activation, and other CAD phenotypes. These and most likely yet-unidentified constituents of blood and plasma determine the rate of atherosclerosis progression.

CAD risk is mainly judged from plasma concentrations of lipids, glucose, and inflammatory markers and from blood pressure, body mass index, and waist-to-hip ratio. Improving lifestyle risk factors, such as smoking, high fat and calorie intake, and lack of exercise, can reduce high blood pressure and body weight, with beneficial effects on risk factors in blood.

Although CAD risk factors are closely interrelated and are monitored lifelong in most people, severe atherosclerosis is usually detected at late stages, often as a result of myocardial infarction (MI), stroke, or other clinical manifestations.

Atherosclerosis is a lifelong, progressive disease that becomes clinically significant in 50% of the population, leading to myocardial infarction and stroke and eventually death. The first manifestation of atherosclerosis is the formation of foam cells in the intima of the arterial wall, leading to the histological appearance of fatty streaks. Briefly, circulating lipoproteins, mainly LDL, adhere to the subendothelial matrix and undergo oxidative modifications that eventually alter gene and protein expression of endothelial cells. These changes lead to the recruitment of monocytes, which migrate to the intima of the arterial wall, differentiate into macrophages, and endocytose the modified LDL. These early steps are followed by additional inflammatory and immune responses, smooth muscle cell migration, and fibrosis, culminating in the formation of atherosclerotic plaques and apoptosis. The interplay of these biological processes, and probably others that have not been identified, underlies the development of atherosclerosis. Lately, statin therapies to lower plasma cholesterol have been shown to prevent or in some cases even regress the development of atherosclerosis (1). However, little is yet known about the repertoire of transcriptional changes underlying atherosclerosis lesion development (2) and scarcely anything about the beneficial effects of plasma cholesterol lowering on arterial wall gene expression.

### SUMMARY OF THE INVENTION

The mapping of the human genome has resulted in a surge of new technologies to study complex diseases like CAD from a genomic perspective. By revealing complete repertoires of molecular activities underlying complex biological systems, these technologies can be used for early identification of disease and new therapies targeting central disease pathways (3-5). In practise this means that research efforts from now on, using these technologies, can identify disease mechanisms from the perspective of all activities leading to the disease and not from the narrow perspective of certain pathways or genes. Since all molecular activities can be monitored, the molecules that are appearing the most central will be the highest ranked as a target for treatment contrasting the history of all targets identified up to date which were pre-selected based on candidate-driven hypothesis. This historical bias is probably why we see so many of today's targets failing in, not seldom, late phases of clinical trials (Phase II and III). The newly identified target genes presented herein represent the new generation targets that are selected based on their high rank in relation to all possible targets for atherosclerosis and atherosclerosis-related diseases.

The target genes presented herein that were found primarily by studies in mice have been identified using a unique mouse model in which plasma cholesterol can be lowered using a genetic switch in the liver that be activated at any given time point in the adult life of the mice (9). Plasma cholesterol lowering is as of today the most efficient way of halting atherosclerosis development. Unfortunately only a small fraction of the population (<10%) is eligible for plasma cholesterol lowering treatments. Using this mouse model, the inventors have identified gene targets that mediated the beneficial effects in preventing atherosclerosis in response to plasma cholesterol-lowering. Hence, these targets can be useful for intervention in the majority of patients who suffers atherosclerosis that also lacks high levels of plasma cholesterol. Developing compounds that directly targets the identified molecules should help to prevent or even to regress atherosclerosis development in these individuals.

The target genes found by studies in humans show that transendothelial migration of leukocytes is a biological process in visceral fat and the arterial wall that contributes to the development of atherosclerosis. This process is general for all inflammatory reactions and thus the identified targets (i.e. genes responsible for this process) may be useful to prevent other inflammatory diseases besides atherosclerosis such as rheumatoid arthritis, inflammatory bowel diseases, Alzheimer to name a few. Another aspect of the study leading to the inventions made in human was that targets were not only sought in the diseased arterial wall (i.e. atherosclerotic arterial wall) but also in the liver, skeletal muscle and visceral fat. This multi-organ screening increases the coverage of putative targets beyond the entire repertoire of molecular activities in the disease itself to all organs that can influence atherosclerosis development. The invention includes 129 genes involved in transendothelial migration of leukocytes (Table 8). The focus for this application is LDB2 which was found to be a high hierarchy regulator of 122 of these 129 genes and thus a suitable target for intervention.

The present invention is based on the discovery of the relation between the genes disclosed in Tables 4 and Table 8, especially LDB2, GYPC, AGPAT3, AGL, PVRL2, HMGB3, HSDL2, and atherosclerosis and atherosclerosis-related diseases.

In a first aspect, the invention relates to a method for identifying a compound as a candidate drug, comprising bringing said compound into contact with a cell expressing a gene selected from the group consisting of the genes disclosed in Tables 4 and Table 8, especially LDB2, GYPC, AGPAT3, AGL, PVRL2, HMGB3, HSDL2, and analyzing if said compound modulates the expression of at least one of said genes.

The modulation in expression may be measured against a reference level in untreated controls by any suitable direct or indirect means available to the skilled person, such as measurement of the amount of transcribed mRNA, amount of produced gene product, activity of gene product or measurement of an introduced reporter entity.

In one embodiment of the invention according to this aspect, the analysis comprises analysis of modulation of expression of at least two of said genes. In a further embodiment, the analysis further comprises analysis of modulation of expression of a gene selected from the group consisting of CD36 and PPARα.

In a second aspect, the invention relates to a method for identifying a compound as a candidate drug, comprising bringing said compound into contact with the gene product of a gene selected from the group consisting of the genes disclosed in Tables 4 and Table 8, especially LDB2, GYPC, AGPAT3, AGL, PVRL2, HMGB3, HSDL2 6, and analyzing if said compound modulates the biological activity of said gene product.

In this aspect, the modulation may be either an increase or a decrease in activity. The activity may be the activity normally associated with said gene product or regulation of expression of a gene implicated in development or progression of atherosclerosis or atherosclerosis-related diseases, such as a gene selected from the group consisting of LDB2, GYPC, AGPAT3, AGL, PVRL2, HMGB3, HSDL2, CD36 and PPARα, or transendothelial migration of leukocytes.

In a further aspect, the invention relates to a method according to any of the previous aspects, comprising
obtaining a DNA molecule comprising the coding sequence of a gene selected from the group consisting of LDB2, GYPC, AGPAT3, AGL, PVRL2, HMGB3, HSDL2, and optionally sequence elements regulating the expression of said gene;
introducing said DNA molecule in a host cell, such as a cell line or a cell of a non-human embryo, to obtain cellular expression of said DNA molecule,
bringing said host cell into contact with said compound, and
analyzing if said compound modulates the expression of said DNA molecule or the biological activity of said gene product.

The analysis step of the method according to this aspect may comprise the analysis of transendothelial migration of leukocytes.

In preferred embodiments of the methods according to the above aspects, the method relates to the identification of a compound as a candidate drug for the treatment of a disease selected from the group consisting of atherosclerosis and atherosclerosis-related diseases.

In the above mentioned aspects, the compound to be identified as a candidate drug may be a small organic molecule, a peptide, polypeptide or protein, a nucleic acid such as DNA or RNA, including siRNA and miRNA, a modified nucleic acid, such as PNA, or any other compound that may be incorporated in a pharmaceutical composition.

In a further aspect, the invention relates to a method for identifying a genetic marker for assessing the predisposition for, development and/or outcome of, atherosclerosis and atherosclerosis-related diseases, such as coronary artery disease, stroke and myocardial infarction, or inflammatory diseases, comprising detecting genetic variations in a gene selected from the group consisting of the genes disclosed in Tables 4 and Table 8, especially LDB2, GYPC, AGPAT3, AGL, PVRL2, HMGB3, HSDL2 between individuals in a population, and correlating said genetic variations to differences in predisposition for, development and/or outcome of, atherosclerosis and atherosclerosis-related diseases between said individuals.

In this aspect of the invention, the genetic variation may be a genetic variation modulating, e.g. increasing or decreasing, either the expression of the gene or the activity of the gene product.

In a further aspect, the invention relates to genetically modified cells and animals comprising a heterologous DNA molecule comprising the coding sequence of a gene selected from the group consisting of the genes disclosed in Tables 4 and Table 8, especially LDB2, GYPC, AGPAT3, AGL, PVRL2, HMGB3, HSDL2 and/or having one of these genes inactivated. Such “knock-out” animals are well known in the art and are produced on request on a commercial basis. The inactivation of the gene need not be 100%; it is sufficient to inactivate the gene to an extent that the phenotype of the knock-out animal is usable in the relevant experiments. It is further possible to introduce a heterologous DNA molecule comprising the coding sequence of the knocked-out gene in the animal, preferably with regulatory sequences that allow the expression of the gene product to be regulated.

In this aspect, the animal may be any non-human animal, preferably a mammal such as a primate or a rodent such as a mouse or rat. The genetically modified cells may be of any origin and the person skilled in the art may decide on a suitable expression system. Genetically modified cells and animals according to this aspect may be used in the above methods for identification of compounds as candidate drugs.

In one embodiment of this aspect, the heterologous DNA molecule further comprises regulatory sequences of the gene selected from the group consisting of the genes disclosed in Tables 4 and Table 8, especially LDB2, GYPC, AGPAT3, AGL, PVRL2, HMGB3, HSDL2.

In a further aspect, the invention relates to a method for treatment of a patient suffering from, or being at risk of developing, atherosclerosis or atherosclerosis-related diseases, such as coronary artery disease, stroke and myocardial infarction, or inflammatory diseases, comprising administering to said patient an original or modified variant of a gene selected from the group consisting of the genes disclosed in Tables 4 and Table 8, especially LDB2, GYPC, AGPAT3, AGL, PVRL2, HMGB3, HSDL2 or a compound identified with the method according to the above mentioned aspects.

In a further aspect the invention relates to a method for treatment of a patient suffering from, or being at risk of developing, atherosclerosis or atherosclerosis-related diseases comprising administering to said patient a compound selected from the group consisting of siRNA molecules targeting a gene selected from the group consisting of LDB2, GYPC, AGPAT3, AGL, PVRL2, HMGB3, HSDL2.

This aspect also covers pharmaceutical compositions comprising siRNA molecules targeting a gene selected from the group consisting of LDB2, GYPC, AGPAT3, AGL, PVRL2, HMGB3, HSDL2 and optionally pharmaceutically acceptable carriers, excipients, diluents and the like. Such siRNA molecules may also be modified for enhanced properties, such as increased uptake, prolonged half-life in vivo etc.

In a further aspect, the invention relates to method for identifying a subject as having an lower than average risk of developing atherosclerosis or atherosclerosis-related diseases, comprising analyzing the LDB2 gene of said subject and wherein the presence of the T minor allele of the single nucleotide polymorphism rs10939673 in the LDB2 gene indicates a lower than average risk.

### BRIEF DESCRIPTION OF THE DRAWINGS

FIG. 1. Atherosclerosis progression in Ldlr^{-/-}Apob^{100/100}Mttp^{flox/flox} mice assessed by Sudan IV staining of pinned-out aortas. Box plots of atherosclerosis progression at 20 (n=12), 30 (n=25), 40 (n=15), 50 (n=15), and 60 weeks (n=10). P<0.05, 20 vs. 30 weeks; P<0.0001, 30 vs. 40 weeks; P<0.02, 40 vs. 50 weeks. Values are surface lesion areas as a percentage of the entire aorta. Boxes enclose values between the 75th and 25th percentiles, bars indicate values between the 90th and 10th percentiles, and black dots indicate individual observations outside these boundaries.

FIG. 2 Relative expression levels of cell-specific markers of atherosclerosis cell types. The number of markers per cell type is indicated. The only statistically significant increase was in the number of foam cells, which increased by 20% between 20 and 30 weeks (P<0.001) and remained elevated at 60 weeks.

FIG. 3: Effect of plasma cholesterol lowering on lesion progression. Lesion surface area was determined by percentage of lesion area in relation to the total area of pinned-out aortas from the bifurcation to the aortic root. At 28 weeks of age, mice received intra peritoneal injections of pI-pC to induce recombination of Mttp in the liver and were sacrificed 12 weeks later or 1 week after cholesterol lowering had been achieved. High-cholesterol control mice were injected with PBS. (A) At 40 weeks of age, lesion surface area in mice with low plasma cholesterol (i.e., pI-pC-treated, n=7) had not progressed and differed significantly from that in high-cholesterol controls at 40 weeks (i.e., PBS-treated, n=6) (*P<0.005). (B,C) One week of low levels of cholesterol (30-week-old mice) did not affect (B) lesion size (P=0.96). Shown are percent relative changes in lesion area. (C) The numbers of foam cells (P=0.52), endothelial cells (P=0.49), smooth muscle cells (P=0.18) (SMC), and T cells (P=0.34)

FIG. 4. A regulatory gene network of foam-cell formation. Twelve cholesterol-responsive atherosclerosis genes were targeted in THP-1 macrophages using siRNA. Two days after transfection, siRNA-targeted macrophages and controls treated with nonspecific siRNA were incubated with AcLDL (50 μg/mL) for 48 hours; total RNA was isolated, and CE and lipid accumulation were determined. (A) Sixteen expression profiles (HG-U133_Plus_{—}2 arrays, Affymetrix) from 12 siRNA experiments and four pooled controls were used to generate the regulatory gene network of 8 cholesterol-responsive genes involved in foam-cell formation, including PPARa and CD36. (B) CE accumulation was decreased by siRNA inhibition of 5 of the 8 genes and increased by inhibition of 2 others; inhibition of 1 gene had no effect (see also Table 2).

FIG. 5. Venn diagrams of clusters related to coronary and carotid stenosis and to the leukocyte transendothelial migration pathway. A. Venn diagrams of genes represented by the clusters in Table 7. Seven genes were found in both the atherosclerotic aortic root/IMA and mediastinal fat clusters (Pc<7×10⁻¹⁰), 17 in the atherosclerotic aortic root/IMA and carotid clusters (Pc<1×10⁻³⁰), and 16 in the mediastinal and carotid clusters (Pc<9×10⁻²⁷). Six genes were found in all three clusters (Pc<7.15×10⁻²³). The union of all three clusters was 129 genes.

### DETAILED DESCRIPTION OF THE INVENTION

The present invention is based on systems biological processing using state of the art cluster algorithms of transcriptional data from mice and humans containing information of the gene activity of all genes in the genome and their activity profile during the development of atherosclerosis. This approach allowed the present inventors to make an unbiased investigation of all genes involved in atherosclerosis, in contrast to the standard technologies in the art wherein an initial selection of interesting genes to study is standard. In other terms, this approach allows the inventors to rank all human genes in order of importance to atherosclerosis. This approach is further explained in the two examples given below.

In example 1, the inventors have shown that lowering of plasma cholesterol before rapid expansion of atherosclerotic lesions prevents further expansion of the atherosclerotic lesions and identified the genes (i.e. targets) that mediated this effect. The bioinformatic methodology (i.e. reversed engineering) used by the inventors made the construction of a gene network of cholesterol-responsive atherosclerosis target genes. The beneficial effects of therapeutic lowering of plasma cholesterol, such as by administration of statins, is mediated through the action of this gene network at least in part. The present invention thus relates to a method for screening of candidate drugs for effects on this plasma cholesterol-regulated gene network as to how it prevent or even regress the development and/or progression of atherosclerosis or atherosclerosis-related diseases. As a validation that the method works, siRNA molecules targeting individual genes in the network were used to modulate expression of the genes. Modulation of gene expression was found to effect the accumulation of cholesterol-esters in macrophages, a process essential to atherosclerosis progression.

In example 2, the inventors used multi-organ whole-genome expression profiling to identify all molecular activities related to atherosclerosis and its related diseases. Using a cluster algorithm that identifies genes with similar expression patterns across the four different organs in all patients, the inventors identified a total of 60 clusters. Of these, 3 were found to be related to the extent of atherosclerosis. These three clusters together represented 129 genes of which a majority had a role in leukocyte migration across the endothelium into diseased tissues. This process was linked to degree of atherosclerosis when active in the arterial wall but also in the mediastinal fat but not in the liver or skeletal muscle. These findings were repeated in a validation cohort of patient suffering atherosclerosis in the carotid arteries (arteries to the head). 122 of the 129 genes were found to have a common regulator; LIM-domain binding 2 (LDB2). This high-hierarchy regulator was thus involved in regulating severity of atherosclerosis and atherosclerosis-related diseases. The T minor allele of the single nucleotide polymorphism, rs10939673, in LDB2 was identified as underrepresented in survivors of myocardial infarction and inversely related to LDB2 mRNA and coronary atherosclerosis. This SNP may thus be utilized as a genetic marker for decreased risk of coronary atherosclerosis.

Since leukocyte migration is an essential path of any inflammatory reaction, the invention of LDB2 as a regulator of this process will have implications as marker and/or as therapeutic target for other inflammatory-related diseases besides atherosclerosis.

The novel gene network and high-hierarchy regulator LDB2 disclosed in the present application also provides new possibilities to identify genetic markers for predisposition for atherosclerosis or atherosclerosis-related diseases, or for predicting the development or outcome of such diseases. Accordingly, the invention partly relates to a method for identification of such genetic markers by comparison of the genotypes of patients with atherosclerosis or atherosclerosis-related diseases with subjects not suffering from such diseases. And, in the case of LDB2, also for other inflammatory-related diseases beside atherosclerosis.

The invention is further described by two investigations of expression profiles, one in mice and one in humans, showing the relation between the identified genes and atherosclerosis and atherosclerosis-related diseases. These investigations serve to illustrate and substantiate the invention and should not be considered as limiting the scope of the invention, which is defined by the appended claims. When practicing the present invention the person skilled in the art may further make of use conventional techniques in the field of pharmaceutical chemistry, immunology, molecular biology, microbiology, cell biology, transgenic animals and recombinant DNA technology, as i.a. disclosed in Sambrook et al. “Molecular cloning: A laboratory manual”, 3^{rd} ed. 2001; Ausubel et al. “Short protocols in molecular biology”, 5^{th} ed. 1995; “Methods in enzymology”, Academic Press, Inc.; MacPherson, Hames and Taylor (eds.). “PCR 2: A practical approach”, 1995; “Harlow and Lane (eds.) “Antibodies, a laboratory manual” 1988; Freshney (ed.) “Culture of animal cells”, 4^{th} ed. 2000; Hogan et al. “Manipulating the Mouse Embryo: A Laboratory Manual”, Cold Spring Harbor Laboratory, 1994; or later editions of these books.

### Example 1

### Transcriptional Profiling and Genetic Lowering of Plasma Cholesterol to Identify Cholesterol-Responsive Atherosclerosis Target Genes

The transcriptional phenotype of atherosclerosis progression is largely unknown. We performed transcriptional profiling of lesion development at 10-week intervals in atherosclerosis-prone mice with human-like hypercholesterolemia and a genetic switch to turn off hepatic lipoprotein production. We show that atherosclerosis progresses slowly at first, expands rapidly after transformation of fatty streaks into plaques, and plateaus after advanced lesions form. The activity of 1259 genes (whereof 329 with previous atherosclerosis relation) forming four distinct expression clusters conveyed this development. Genetic lowering of plasma cholesterol in mice with early lesions resulted in a distinct transcriptional response, preventing the rapid expansion and the transformation into plaques. 37 cholesterol-responsive genes (Table 4) were identified whereof >90% with no previous relation to atherosclerosis. In six silencing interfering RNA mediated inhibitions of a total of ten cholesterol-responsive genes, the generation of foam cells from THP-1 macrophages was also affected. Thus, by careful investigation of the transcriptional phenotypes of lesion progression and its prevention upon lowering of plasma cholesterol, cholesterol-responsive atherosclerosis target genes could be identified.

Atherosclerosis is a lifelong, progressive disease that becomes clinically significant in 50% of the population, leading to myocardial infarction and stroke and eventually death. Lately, statin therapies to lower plasma cholesterol have been shown to prevent or in some cases even regress the development of atherosclerosis. However, little is yet known about the repertoire of transcriptional changes underlying atherosclerosis lesion development and scarcely nothing about the beneficial effects of plasma cholesterol lowering on arterial wall gene expression. Whole-genome measurement technologies developed in the aftermath of the human (6, 7) and mouse (8) genome projects now offer the opportunity to elucidate the entire repertoire of expression changes in relation to complex diseases like atherosclerosis. We studied the Ldlr^{-/-} Apob^{100/100}Mttp^{flox/flox}Mx1-Cre mouse model (9) to investigate lesion progression, the underlying transcriptional phenotypes and the effects of plasma cholesterol lowering. These mice have a plasma lipoprotein profile similar to that of familial hypercholesterolemia (Ldlr^{-/-}Apob^{100/100}) and contain a genetic switch to turn off hepatic synthesis of lipoproteins (Mttp^{flox/flox}Mx1-Cre).

### Atherosclerosis Progression

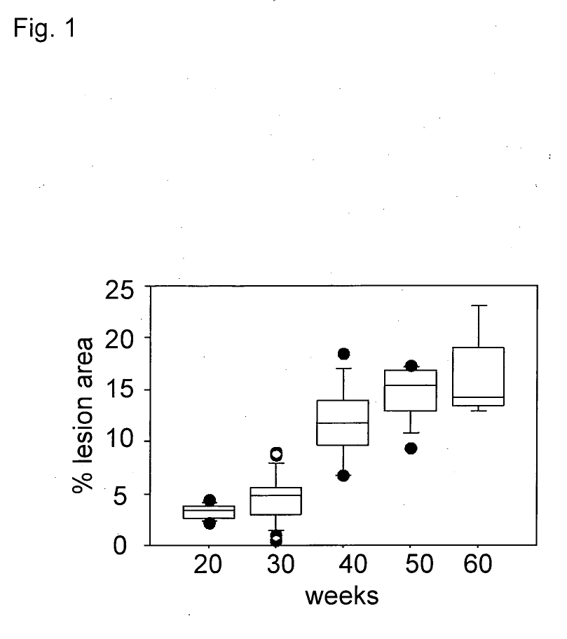

Mice were examined at 10, 20, 30, 40, 50, and 60 weeks of age. Plasma cholesterol increased slightly over time, but triglyceride and glucose levels did not change significantly (Table 1). Lesion area and morphological changes were assessed in 87 Ldlr^{-/-}Apob^{100/100}Mttp^{flox/flox} mice (FIG. 1). Only occasional spots of Sudan IV staining were detected at 10 weeks (n=10, not shown), but lesions were detected in all mice at 20 weeks. Lesion size increased by ˜1.6% between weeks 20 and 30 (P=0.05) and by ˜7.2% between weeks 30 and 40 (P<0.0001). At 20 weeks, the main morphological feature was fatty streaks (red transparent areas with diffuse boundaries); no plaques were detected. However, at 30 weeks, all mice had small plaques (red nontransparent areas with distinct boundaries) in the aortic arch that had expanded substantially by 40 weeks. At 50 and 60 weeks, plaque growth was restrained. The development of lesions over time was mirrored by changes in Oil Red O staining and fluorescence from the CD68 antigen (not shown).

### Transcriptional Phenotype of Atherosclerosis Progression

Next, we identified transcriptional changes underlying the histological changes during atherosclerosis progression. Of 19,879 genes in the Mouse Genome Informatics Database (see www.jax.org), 6.3% were differentially expressed in at least one time comparison (FDR<0.05, uncorrected P<0.00008, n=1259), and 329 (27%) had previously been related to atherosclerosis. Of the remaining 73%, 95% had known biological function according to GO analyses. Of genes with established roles in atherosclerosis, 78% were differentially expressed in at least one time comparison (P<0.05, n=88/111).

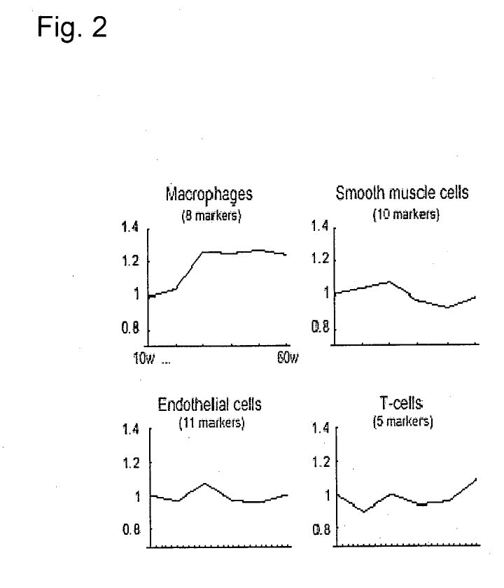

To reveal gene expressional patterns during atherosclerosis progression, we performed cluster analysis of mRNA levels of the 1259 differentially expressed genes. Four distinct clusters were generated (Table 3). Genes in cluster 1 (n=293) were activated during the rapid expansion of the lesions (FIG. 1), remained activated throughout 60 weeks, and had the highest percentage of genes previously related to atherosclerosis and atherosclerosis cell types (Table 3). Of the genes in cluster 1, 89% were related to inflammatory cells, including the macrophage-marker CD68, which increased fivefold; increased CD68 expression was also identified immunohistochemically (not shown). These findings together with an increase in the relative number of 8 macrophages markers (FIG. 2), strongly suggest that the expression pattern in cluster 1 reflected the recruitment and activation of lesion macrophages.

Gene activities peaked at week 30 in cluster 2 (n=331) and at week 40 in cluster 4 (n=300) and were suppressed at the late stages of atherosclerosis progression. Of the genes in these clusters, 73% had no previous relation to atherosclerosis or atherosclerosis cell types. Of 20 transcription factors, TFs, 17 were deactivated and only three were activated. The functional annotations of clusters 2 and 4 indicate possible involvement in the proliferation and migration of smooth muscle cells into the lesion. Cluster 3 (n=339) was particularly revealing. The mRNA levels of these genes peaked at 30 weeks and were suppressed at 40 weeks, coinciding with the rapid expansion of atherosclerotic lesions. Moreover, this cluster contained fewer atherosclerosis-related genes than cluster 1 but more than clusters 2 or 4 and consisted mainly of genes related to carboxylic and lipid metabolism. Thirteen of 19 TFs in this cluster were well-established in lipid and energy metabolism, such as the peroxisome proliferator activator receptors (PPARs) PPARa, PPARd, and PPARγ and sterol regulatory element binding factor 2. Apoptosis and cell death were active processes in clusters 2 to 4 but not in cluster 1. This finding is consistent with the notion that cell death and apoptosis are continuous processes during all phases of atherosclerosis development(10).

### Transcriptional Phenotype of Foam Cells

Apart from foam cells, which increased in number between weeks 20 and 30 (P<0.001; FIG. 2), the relative amounts of the other major lesion cell types (i.e., endothelial, smooth muscle, and Tcells) were relatively stable over time, as indicated by the mRNA levels of cell-type-specific gene markers.

The recruitment of lipid-poor macrophages at 30 weeks that expanded and became lipid-enriched at 40 weeks was verified immunohistochemically.

In summary, the gene expression data suggest that lipid-poor macrophages gradually accumulate in the early phases of lesion development, reaching a critical mass at 30 weeks (FIG. 2), inducing an inflammatory reaction (Table 3, cluster 1), increasing lipid accumulation in foam cells (Table 3, cluster 3), and causing rapid lesion expansion (FIG. 1). The lipid accumulation in foam cells was sustained until 40 weeks (Table 3, cluster 3). The inflammation persisted in the later phases (Table 3, cluster 1). Clusters 2 and 4 are novel in relation to atherosclerosis (Table 3).

### Effects of LDL Cholesterol Lowering on Atherosclerosis Progression

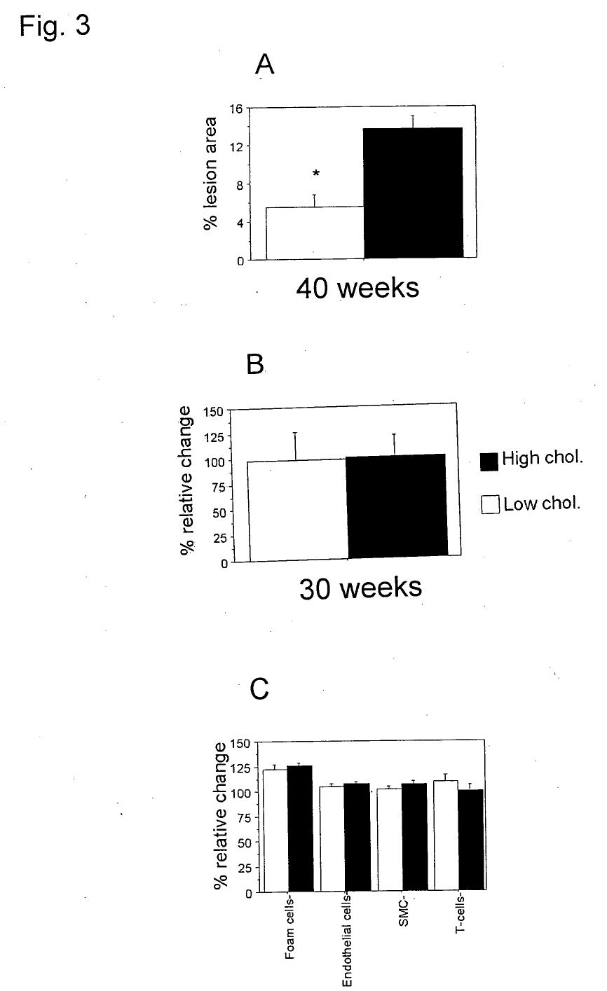

Next, we genetically lowered plasma LDL cholesterol in 30-week-old mice by treatment with polyinosinic polycytodylic acid (pI-pC) to induce the Mx1-promoter of the Cre transgene mainly in the liver, resulting in the recombination of Mttp (Ldlr^{-/-}Apob^{100/100}Mttp^{Δ/Δ}); control mice were treated with saline. We chose the 30 week time point because it preceded the rapid expansion of the atherosclerotic lesions and because of the expression patterns of the identified atherosclerosis genes in the lesions and foam cells over time. Plasma cholesterol levels were lowered by more than 80% upon recombination of Mttp (from 427 to 54±31 mg/L, n=4) and remained at this level for 10 weeks until sacrifice. At sacrifice, the lesion size in these mice had not increased and was significantly less than in 40 weeks old mice with high cholesterol (FIG. 3A, P<0.005). Thus, the lowering of plasma cholesterol at 30 weeks prevented the rapid expansion of atherosclerotic lesions observed in mice with high plasma cholesterol levels (˜7.2%, P<0.0001; Table 1 and FIG. 1).

### Effects of Cholesterol Lowering on the Transcriptional Phenotype of Atherosclerosis

To identify the transcriptional changes induced by plasma cholesterol lowering, we again recombined hepatic Mttp in 28-week-old mice, but this time sacrificed the animals just 1 week after the cholesterol lowering had been accomplished (at 30 weeks). Before examining gene expression changes in lesions, we examined other possible sources affecting lesion expression. First, we examined whether lesion size and the relative numbers of the four major cell types differed in pI-pC-treated mice with lowered cholesterol (Mttp recombined) and saline-treated controls with high cholesterol (Mttp intact). There were no differences in lesion size (FIG. 3B) or in cell type numbers (FIG. 3C), as indicated by the expression levels of cell type-specific gene markers. These observations were confirmed by the histological appearance of Oil Red O-stained lesions and immunohistochemical analysis of lesions for CD68 expression (not shown). 37 of the most significantly changed plasma-cholesterol-responsive genes in the atherosclerotic arterial wall are shown in Table 4.

To exclude the possibility that any gene expression changes resulted from hepatic activation of the Mx-1 Cre-transgene rather than cholesterol lowering, we bred mice lacking the lox P sites flanking the Mttp promoter and exon 1 (Ldlr^{-/-}Apob^{100/100}Mttp^{wt/wt}) and performed transcriptional profiling of lesion RNA isolated from pI-pC-treated and PBS-treated mice (n=5 each). None of the genes in Table 4 were among the few significantly changed arterial wall genes identified in this comparison (data not shown).

Effects of Gene Silencing on Foam Cell Formation from Cultured THP-1 Macrophages

For several reasons, foam cell formation appeared to have a crucial role in the rapid expansion of lesions between weeks 30 and 40. First, the rapid expansion of lesions was preceded by accumulation of macrophages in the arterial wall at 30 weeks (FIG. 2). Second, lesion cluster 3, which contained genes of importance for intracellular lipid metabolism, was transiently activated at 30 weeks. Third, inflammatory and immune responses were activated at 30 weeks in lesion cluster 1. Fourth, and perhaps most importantly, TFs with established roles in inflammation and lipid homeostasis in foam cells were deactivated at 40 weeks (PPARs and SREBP-2).

The rapid expansion of the lesions from weeks 30 to 40 was primarily caused by lipid loading of foam cells present at 30 weeks (clusters 1 and 3 in Table 2). We suspected that some of the cholesterol-responsive genes identified at 30 weeks (Table 4) were essential to this process. To address this, we selected 12 of the 37 genes that previously had been related to foam-cell formation or were known macrophage genes (see Methods). These 12 genes were targeted by siRNA in THP-1 macrophages incubated with AcLDL. mRNA from the targeted cells was subjected to transcriptional profiling (n=15), and the regulatory gene network of foam-cell formation was inferred as described(1) (see Methods).

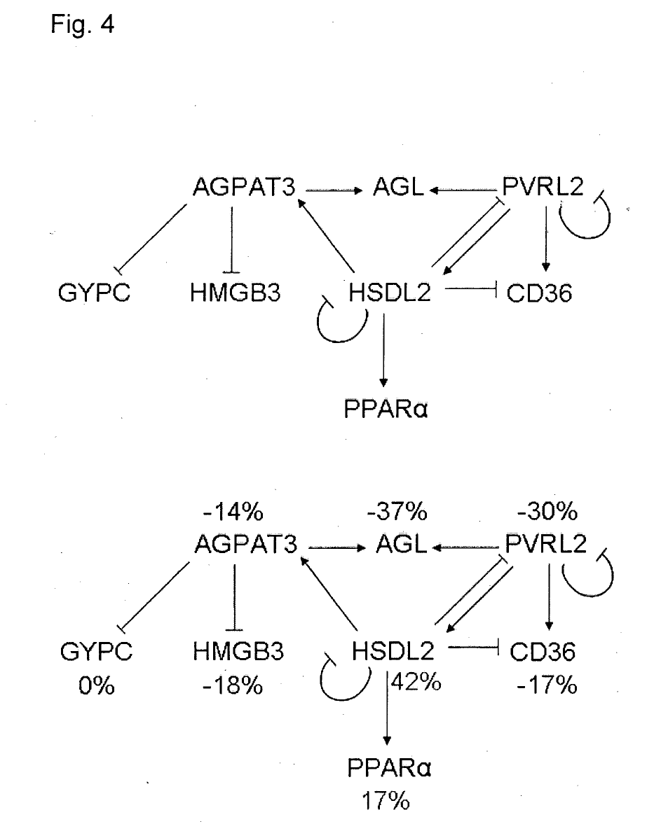

Eight of the targeted genes belonged to a common regulatory gene network (FIG. 4A) and were efficiently inhibited by siRNA (Table 2). These genes included CD36, which promotes foam-cell formation, and PPARa, which prevents it. Hydroxysteroid dehydrogenase-like 2 (HDSL2) up-regulated PPARa and down-regulated CD36. Moreover, poliovirus receptor-related 2 (PVRL2) also regulated CD36, increasing its expression and negatively regulating HSDL2 and thus indirectly suppressing PPARa activity. From the regulation of CD36 and PPARa in this regulatory network (FIG. 4A), we predicted that inhibiting PVRL2 would prevent foam-cell formation and inhibiting HDSL2 would promote it. To test these predictions and to assess the effect of inhibiting individual genes within the network, we measured cholesterol-ester (CE) and lipid accumulation (FIG. 4B and Table 2). In general, the CE measurements confirmed our predictions and showed that several of the other network genes also affected CE accumulation in THP-1 macrophages (FIG. 4B, Table 2).

In this study, we identified a critical point before which atherosclerosis developed slowly and only low-level inflammation was present. Thereafter, lesions expanded rapidly and inflammation increased markedly. The inflammation persisted in late phases, leading to the formation of advanced plaques, but lesion size increased transiently during a 10-week period. The rapid lesion expansion was primarily caused by an equally rapid CE accumulation in macrophages. Macrophages with a low content of lipids accumulated in the early phases of atherosclerosis development, reaching a critical density that initiated the rapid accumulation of lipids and inflammation. Lowering plasma cholesterol at this critical point prevented the rapid expansion of the lesions and the formation of advanced plaques. The cholesterol-lowering effect was mediated at least in part by 37 cholesterol-responsive atherosclerosis genes. Validation of some of these genes by transcriptional profiling of siRNA-targeted THP1-macrophages incubated with AcLDL exposed a regulatory gene network of foam-cell formation. The architecture of this network highlighted PVRL2 and HSDL2 as novel candidate genes that might be good targets for future therapies to prevent the formation of advanced plaques.

Transcriptional profiles of atherosclerosis lesions are challenging to interpret. (2) Such lesions contains several cell types, and the average mRNA contribution of a given cell type is altered with disease development. Thus, changes in mRNA levels represent a mixture of actual changes in cellular mRNA concentrations and changes in cell type admixture. In addition, cells in different stages of proliferation and differentiation (e.g., macrophages differentiation into foam cells) adds to this problem also within a given cell type. However, the lesion mRNA concentrations provide a general picture of the biological processes and pathways activated in the lesion.

Analysis of lesion mRNA clusters (Table 3) indicated that, at first, macrophages relatively slowly infiltrate the arterial wall, leading to the formation of fatty streaks. Then, at what appears to be a rather specific time point, these cells become activated, leading to a burst of inflammatory activity that, in combination with a rapid accumulation of CE in macrophages, generates advanced plaques. We believe this transformation can be related to the density of macrophages in the arterial wall. At a given density, the macrophages not only stimulate themselves (autocrine) but also stimulate each other (paracrine), leading to a burst of inflammatory activities and increasing lipid uptake. (12) If such a mechanism is also present in humans, the timing of therapies to prevent or slow atherosclerosis development may be very important. Indeed, in mice, the formation of advanced plaques was prevented by genetic lowering just before the rapid lesion expansion (FIG. 3A).

In contrast to lesion development, the extent and relative composition of different cell types in the lesions were similar before and after the subacute lowering of plasma cholesterol (FIGS. 3B, C). Thus, the changes in mRNA concentration monitored by the GeneChips are likely to reflect actual changes in cellular mRNA levels. Since the accumulation of lipids in macrophages was a central process in the rapid lesion expansion, 12 of 37 cholesterol-responsive genes were validated by siRNA in THP-1 macrophages incubated with AcLDL. Eight genes were found to belong to a common regulatory gene network in which PVRL2 and HSDL2 had central roles. Little is known about PVRL2 and HSDL2 (generating 6 and 3 hits in PubMed, respectively). A sequence variant in PVRL2 is associated with the severity of multiple sclerosis. (13) HSDL encodes the sterol carrier protein-2, a small intracellular basic protein domain that enhances the transfer of lipids between membranes in vitro. (14) As indicated by the regulatory gene network (FIG. 4A), none of the nodes (i.e., genes) in the regulatory network solely promoted or inhibited foam-cell formation, highlighting the importance of inferring gene networks to understand and evaluate the true complexity of candidate genes in complex diseases. (11, 15)

Our findings imply that the timing of interventions with plasma cholesterol-lowering agents is critical. Patients at risk of developing complications of atherosclerosis (e.g., stroke and myocardial infarction) may benefit from being treated very early in life. Noninvasive technologies to detect early atherosclerosis are important in this respect. For normocholesterolemic individuals who have other atherosclerosis risk factors, novel regimens targeting atherosclerosis genes that mediate the beneficial effects of plasma cholesterol-lowering may be useful.

Accordingly, one aspect of the present invention is to identify compounds as candidate drugs for future therapies to prevent development of late atherosclerosis lesions, which compounds target these cholesterol-responsive genes. This aspect is further defined in the appended claims.

### Methods

### The Mouse Model

The Ldlr^{-/-} Apob^{100/100}Mttp^{flox/flox}Mx1-Cre mouse model has a plasma lipoprotein profile similar to that of familial hypercholesterolemia, which causes rapid progression of atherosclerosis. (9) For Mttp deletion, mice were injected with 500 μl of pI-pC (1 μg/μl; Sigma, St. Louis, Mo.) every other day for 6 days to induce Cre expression, thereby recombining Mttp (Mttp^{Δ/Δ}) or not in the Ldlr^{-/-}Apob^{100/100}Mttp^{wt/wt}Mx1-Cre mice. Littermate controls received PBS (Mttp^{flox/flox}). The study mice had been back crossed 5 times to C57BL/6 (<5% 129/SvJae and >95% C57BL/6), were housed in a pathogen-free barrier facility (12-hour light/12-hour dark cycle), and were fed rodent chow containing 4% fat. Genotypes were determined by polymerase chain reaction (PCR) with genomic DNA from tail biopsies. Plasma cholesterol and triglyceride concentrations were determined with calorimetric assays (Infinity cholesterol/triglyceride kits; Thermo Trace), and plasma glucose levels with Precision Xtra (MediScience, Cherry Hill, N.J.).

### En Face Analysis and Histology

Aortas were pinned out flat on black wax surfaces as described, (16) stained with Sudan IV, photographed with a Nikon SMZ1000 microscope, and analyzed with Easy Image Analysis 2000 software (Telmo Optik, Skarholmen, Sweden). Lesion area was calculated as a percentage of the entire aortic surface between the aortic root and the iliac bifurcation. Aortic roots were isolated and immediately frozen in liquid nitrogen in OCT compound (Histolab, Västra Frölunda, Sweden). Cryosections (20 μm) were cut and stained with hematoxylin and Oil Red O as described; (17) other sections (6-8 μm) were incubated first with rat anti-mouse CD68 antibody or a control antibody (Serotec) overnight at 4° C. and then with fluorescent anti-rat IgG (Vector Laboratories, Burlingame, Calif.) and counterstained with mounting medium containing DAPI (Vector Laboratories).

### Transcriptional Profiling

Aortas were perfused with RNAlater (Qiagen, Valencia, Calif.), and the aortic arch from above the third rib to the aortic root was removed and homogenized with FastPrep (Qbiogene, Irvine, Calif.). Total RNA was isolated with RNeasy Mini Kit (Qiagen) using a DNAse I treatment step. RNA quality was assessed with a Bioanalyzer 2100 (Agilent Technologies, Santa Clara, Calif.). High-quality RNA samples (32 from Mttp^{flox/flox}, 5 from Mttp^{Δ/Δ}, and 9 Mttp^{wt/wt} mice) were used for global gene expression measurements with cDNA arrays (Mouse Genome 430 2.0 GeneChips, Affymetrix, Santa Clara, Calif.) at 10 (n=7), 20 (n=5), 30 (n=6+5) (pI-pC) and 9 Mttp^{wt/wt} (n=5 (PBS)+4 (pI-pC)), 40 (n=5), 50 (n=5), and 60 (n=4) weeks. All samples were prepared with the two-cycle protocol recommended by the manufacturer. Arrays were scanned with GeneChip Scanner 3000 and analyzed with GeneChip Operating Software (Affymetrix).

### Text Mining and Prior Atherosclerosis Knowledge

Automated text mining of PubMed was used to establish lists of genes related to atherosclerosis, foam cells, smooth muscle cells, endothelial cells, and T cells. Briefly, a gene was considered related if it co-occurred with any of the following terms in the abstract of an article in PubMed: atherosclerosis, arteriosclerosis (“atherosclerosis-related”), foam cell, macrophage, monocyte (“foam cell related”), smooth muscle cell, endothelial cell, and T cell. These hits constitute fairly comprehensive but not specific lists of genes with possible roles in atherosclerosis or in the cell types involved in atherosclerosis (i.e., may contain false positives but a low number of false negatives) with a substantial overlap. We also generated a list of “established” atherosclerosis genes by manually extracting from recent reviews genes known to be important in atherosclerosis.

siRNA of THP-1 Macrophages Incubated with Acetylated LDL

Monocytes of the human monocytic cell line THP-1 were plated in six-well culture dishes (Falcon, Becton Dickinson Labware) at 6×10⁵ cells/well in 10% fetal calf serum (FCS)-RPMI-1640 medium with L-glutamine (2 mM) and HEPES buffer (25 mM) (Gibco-Invitrogen, Carlsbad, Calif.) supplemented with penicillin (100 U/mL) and streptomycin (100 μg/mL) (PEST) and induced to differentiate into macrophages with phorbol 12-myristate 13-acetate (PMA)

(50 ng/mL) (Sigma) for 72 hours. For each gene, cells were transfected with up to three siRNAs (Ambion, Austin, Tex.), using Lipofectamine 2000 according to the manufacturer's instructions (Invitrogen), in medium without FCS, PEST, and PMA. Two days after transfection, siRNA-targeted macrophages and mock-treated controls (nonspecific siRNA) were incubated with acetylated LDL (AcLDL, 50 μg/mL) for 48 hours in 1% FCS medium with PEST. AcLDL was prepared as described. (18) The samples were dialyzed against PBS at 4° C. AcLDL protein concentration was determined by the Bradford method. LDL was isolated from the plasma of healthy donors by sequential ultracentrifugation. (19)

### Lipid, Protein, and Gene Expression Measurements

For lipid imaging, THP-1-derived foam cells were fixed with 10% formaldehyde in PBS for 10 min and washed twice with PBS. The cells were stained with Oil Red O (0.3% in 60% isopropanol) for 20 min, washed twice with 60% isopropanol and twice with PBS, and examined with a Nikon Eclipse E800 microscope at 40× magnification. Lipids were isolated by hexan/isopropanol (3:2) extraction at room temperature for 1 hour followed by 0.5 ml chloroform for 15 min(20). The lipid extracts were dried and resuspended in 80 μl of isopropanol with 1% Triton-X-100 (Sigma). The lipid content of the foam cells was determined by enzymatic assays using the Infinity kit for total cholesterol (Thermo Trace) and a kit for free cholesterol (Wako Chemicals, Richmond, Va.). After lipid extraction, proteins were extracted from the same wells by incubation with 0.5 M sodium hydroxide for 5 hours at 37° C. Protein concentration was determined by the Bradford method.

For HG-U133_Plus_{—}2 array analysis (Affymetrix) and to determine the degree of knockdown by siRNA, total RNA was isolated from the AcLDL-incubated THP-1 cells with RNeasy Mini-kit (Qiagen). The concentration was determined with a spectrophotometer (ND-100, NanoDrop Technologies, Wilmington, Del.). For cDNA synthesis, 0.5 μg of total RNA was reverse transcribed with Superscript II (Invitrogen) according to the manufacturer's protocol. After 5-fold dilution, cDNA (3 μL) was amplified by real-time PCR with 1× TaqMan universal PCR master mix (Applied Biosystems, Foster City, Calif.) on an ABI Prism 7000 (PE Biosystems) and software according to the manufacturer's protocol. Assay-On-Demand Kits containing corresponding primers and probes from Applied Biosystems were used, and expression values were normalized to acidic ribosomal phosphoprotein P0. Each sample was analyzed in duplicate.

### Statistics and Calculations

Differences in the mRNA levels of selected genes, mouse plasma measurements, and lesion surface areas between time points were analyzed with unpaired t tests. Gene expression signal-level data were computed with MAS 5.0 (Affymetrix) using default settings, log-transformed, and normalized to total intensities (global scaling). After normalization, signal intensities were computed for each gene in the Mouse Genome Informatics Database (MGD genes, Jackson Laboratory, www.jax.org) by averaging the signal of the corresponding Affymetrix probe sets. Of the 11,979 GeneChip probe sets (Mouse Genome 430 2.0 GeneChips, Affymetrix) that had no match in the database, 1.5% were differentially expressed (false discover rate (FDR)<0.05, n=177), representing the fraction of genes/probe sets that were not considered for further analyses. The remaining 33,122 probe sets had at least one match in 19,879 MGD genes (of a total of 32,095). Lowess normalization (21) was applied in pair-wise fashion before differential expression testing. To correct for multiple testing when computing probabilities of differential expression and FDRs, we used empirical Bayes statistics. (22) Clustering was performed with the FindCluster algorithm in Mathematica 5.1 (Wolfram Research, Champaign, Ill.). GO and pathway analyses were performed with EASE software. (23) The regulatory gene network of THP-1 macrophages incubated with AcLDL was inferred as described (11) (see below).

### Cholesterol-Responsive Atherosclerosis Genes Identification

Cholesterol-responsive atherosclerosis gene were considered those genes that were differently expressed (FDR<0.05) in the atherosclerotic aortic arch of mice in which Mttp recombination in the liver (see above) had been induced by intra peritoneal injections with 500 μl pIpC (1 μg/μl) compared to PBS injected controls (see Table 4, n=37). The injections were performed at four sequential time points with two days interval starting at the first day of week 29 weeks continuing until the end of week 29. pIpC-treatment achieved a lowering of plasma cholesterol with 80% or more in all mice as measured in plasma at sacrifice at 30 weeks. Plasma cholesterol levels in control mice treated with saline were unaffected. During week 30, the mice were left alone to wash out any remaining effects of the injections. None of these 37 genes were identified in pIpC-treated control mice lacking the floxed Mttp (Ldlr^{-/-}Apob^{100/100}Mttp^{wt/wt}Mx1-Cre) and thus, the recombination of Mttp did no take place nor did the plasma cholesterol lowering. These control mice were investigated with gene chip arrays to exclude the possibility that the plasma cholesterol-responsive genes instead were pIpC induced genes in the atherosclerotic lesions.

Cholesterol-Responsive Genes Selected for siRNA Targeting

Of the 37 identified cholesterol-responsive genes, 27 had preidentified TaqMan and siRNA assays (Invitrogen). In table 4, 12 of these 37 genes are marked in bold indicating that they previously have been reported as expressed by THP-1 macrophages. These were targeted by silencing interfering RNA. Among these were CD36 included as positive control1 and PPAR-a as negative control2.

### Regulatory Network Identification

Expression data (Affymetrix Hu 130, 2.0+) was generated from 12 siRNA experiments (>58% inhibition for all experiments, see also Table 4, genes marked in bold) and 4 pools of controls treated with unspecific siRNA (mock). Total RNA was isolated from targeted and control THP-1 macrophages in cell culture that had been activated by PMA, treated with siRNA or mock and then incubated with acetylated LDL for 48 hours (see also above). Three transcripts, GPR120, GPR81 and SOX6, were below the detection limits of the GeneChips suggesting that these genes were not active enough in this experimental model of foam cell formation to be detected or inactive. The remaining genes were organized in a 9-by-9 data matrix. Expression data for each gene was normalized by dividing with the mean expression level in controls followed by log-transformation. A linear gene regulation model

$$\frac{d {x}_{i}}{d t} = {\sum }_{j = 1}^{n} {w}_{ij} {x}_{j} + {p}_{i}$$

was fit to data as previously described (11). Here x denotes expression data vectors, W is the network adjacency matrix, and p is the perturbation vector. In each knockdown experiment, the elements of p were −1 for the perturbed gene and 0 for all other genes. Note that because of the log-transform, this corresponds to a multiplicative model in actual expression levels. The algorithm controls the tradeoff between precision and recall by a single parameter d. In our experiments we chose d=0.2. In simulations we found that this value corresponds to approx. 60% precision and 80% recall. Of note, interactions between genes (i.e. directed edges with stimulating or repressing effect) do not imply direct biological interactions but in most instances indirect (for instances mediated by proteins, metabolites or even intermediate genes (with low expression level such as transcription factors)).

### Example 2

### Multi-Organ Gene Expression Profiling Indicates Novel Candidate Genes in Coronary Artery Disease

In this example we performed multi-organ gene expression profiling in patients with coronary artery disease (CAD) in the Stockholm Atherosclerosis Gene Expression (STAGE) study.

### Methods

In the STAGE study, Affymetrix HG-U133 GeneChips were used to obtain 278 transcription profiles from atherosclerotic and unaffected arterial wall (n=40×2) and from liver, skeletal muscle, and mediastinal fat (n=66×3) during coronary artery bypass grafting. A validation cohort (25 carotid stenosis patients) was also analyzed. Clusters of mRNA levels were identified by coupled two-way clustering.

### Patients and Biopsy Collection

To explore new CAD and atherosclerosis expression phenotypes, 124 patients admitted for CABG (=2 grafts) at the Karolinska University Hospital, Solna were included in the STAGE study. Forty-two patients undergoing carotid surgery at Stockholm Söder Hospital were recruited as a validation cohort. The exclusion criteria were other severe diseases (e.g., cancer, kidney disease, and chronic systemic inflammatory diseases). The studies were approved by the Ethics Committee of the Karolinska University Hospital, Solna. All patients gave informed consent. A genetic validation was performed in 387 MI survivors with matched controls <60 years of age (39) and in 1091 MI survivors with matched controls of the Stockholm Heart Epidemiology Program (SHEEP). (24).

Four surgeons performed the CABG, and two the carotid surgery. Anaesthesia was standardized; systolic blood pressure was kept at <150 mmHg. In CABG patients, biopsies were obtained from the internal mammary artery (IMA), aortic root, liver, skeletal muscle, and mediastinal fat, preserved in RNAlater (Qiagen) and frozen at −80° C. The presence of atherosclerotic lesions in the aortic root samples (25, 26) and the absence of lesions in the IMA (27) were confirmed by macroscopic and microscopic examinations (not shown). Carotid plaques were dissected from the arterial wall, minced, washed with RNase-free water, embedded in OCT medium (Tissue-Tek, Histolab Products), frozen in liquid isopentane and dry ice, and stored at −80° C.

### Follow-Up Visit and Laboratory Measurements

One hundred fourteen of 124 CABG and thirty-nine of 42 carotid stenosis patients came to a 3 month follow-up visit. Using a standard questionnaire, a research nurse obtained a medical history and information on lifestyle factors (e.g., smoking, alcohol consumption, and physical activity). A physical examination was performed, and venous blood samples were drawn into precooled sterile tubes (Vacutainer, Becton Dickinson) containing NaEDTA and placed on ice. Plasma was recovered within 30 minutes by centrifugation (2.750 g, 20 minutes, 4° C.) for analysis of cholesterol, triglyceride, and lipoproteins as described (28). Blood glucose was measured by a glucose oxidase method (Kodak Ektachem) and insulin and pro-insulin by enzyme-linked immunosorbent assay (Dako Diagnostics).

### RNA Isolation and Expression Profiling

Total RNA was isolated with Trizol (BRL-Life Technologies) and FastPrep (MP Biomedicals), purified with RNeasy Mini kit (Qiagen), and treated with RNase-Free DNase Set (Qiagen). Sample quality was assessed with an Agilent Bioanalyzer 2100. cRNA yield was assessed with a spectrophotometer (ND-1000, NanoDrop Technologies) before hybridization to HG-U133_Plus_{—}2 arrays (Affymetrix). The arrays were processed with a Fluidics Station 450, scanned with a GeneArray Scanner 3000, and analyzed with GeneChip Operational Software

2.0. Expression profiling was performed on all five biopsies in 40 patients, on the three metabolic biopsies in an additional 26 patients from the STAGE study, and on carotid lesions from 25 randomly selected carotid stenosis patients.

### Coronary and Carotid Atherosclerosis Measurements

All CABG patients underwent preoperative biplane coronary angiography (Judkins technique). Angiograms were evaluated with quantitative coronary angiography (QCA) techniques (Medis). The left and right coronary arteries and their branches were divided into segments (29). Each segment was measured during end-diastole, and plaque area determined as a percentage of total area of the segment. Some patients had right coronary artery occlusion that prohibited QCA evaluation. A coronary stenosis score was calculated from all atherosclerotic lesions in the coronary arteries (1 and 2 point(s) for 20-50% and >50% obstruction of the lumen, respectively).

Before surgery, carotid arteries were examined with B-mode ultrasound. The far wall of the common carotid artery was used for measurements of intima-media thickness (IMT) from the endoarterectomy side (30). (30)

### Genotyping

DNA was extracted from blood with Qiagen Blood and Cell Culture DNA kits. Genotyping was performed with TaqMan SNP Genotyping Assays (Applied Biosystems). Five single-nucleotide polymorphisms (SNPs), evenly distributed in different linkage disequilibrium (LD) blocks according to SNPbrowser Software 3.5 (Applied Biosystems), were selected in the LIM-domain binding 2 (LDB2) gene (dbSNP: rs872478, rs1501127, rs10939673, rs2658509 and rs7671482).

0.5 μg of total RNA was reverse transcribed with Superscript II (Invitrogen) according to the manufacturer's protocol. After 5-fold dilution, cDNA (3 μL) was amplified by real-time PCR with 1× TaqMan universal PCR master mix (Applied Biosystems) on an ABI Prism 7000 (PE Biosystems) and software according to the manufacturer's protocol. The Assay On-Demand Kits containing corresponding primers and probes from Applied Biosystem were used. mRNA levels were normalized to 36B4. Each sample was analyzed in duplicate.

### Calculations and Statistical Analyses

Clinical and metabolic characteristics are given as continuous variables with means±SD and as categorical variables with numbers and percentages of subjects. P values were calculated with unpaired t tests; skewed values were log-transformed. For SNP analyses, ANOVA, chi-square, and logistic regression (StatView 5.0.1) were used. Gene expression values were pre-processed Quantile Normalization and the Robust Multichip Average (31) (see also Supplementary Methods) of 604,258 perfect-match Affymetrix probe signals, 423,636 could be mapped to refseq transcripts (32), generating 15,042 refseq transcripts. Gene expression data were clustered by a coupled two-way approach (33, 34)First, genes clusters were identified with a super paramagnetic clustering algorithm (33). Second, for each gene cluster, patients were grouped by hierarchical clustering (35) (see Supplementary Methods). Clusters were visualized with TreeView (35). Probabilities of differential expression and false discovery rates were computed as described (22) Gene Ontology (GO) and pathway analyses were performed with DAVID software (56) and all calculations with Mathematica 5.1. Text mining was used to define transcripts previously related to CAD and atherosclerosis (see Supplementary Methods). For the promoter analysis, TRANSFAC (36)was used.

### Supplementary Methods

### Biopsy Collection

The 114 patients included in the STAGE study underwent isolated elective coronary artery by-pass grafting (CABG). Five tissue samples were obtained during the operation. 0.5 g of skeletal muscle was taken from the medial border of the apical rectus abdominis muscle close to the incision and about 1 g of mediastinal fat from the tissue anterior to the pericardium and great vessels. The internal mammary artery was dissected from the inside of the left chest wall and 1 cm of the distal part was cut. Full thickness aortic wall samples were obtained from the hole punch used to create the proximal vein graft anastomoses at the aortic root during the operation. About 0.05 g of liver tissue (3 mm in diameter) was taken from the very inferior border of the left liver lobe at the end of the operation. This part of the liver was easily accessed after the peritoneum was opened a few centimeters just below the xiphoid process. The minimal incision was sutured after removal of the biopsy and the peritoneum was again closed. All tissue samples were taken without use of cautery and without complications. They were put immediately into RNAlater (Qiagen) solution within 10 seconds and frozen at −80C until further processing.

### Cluster Analysis

Gene expression data from each tissue was clustered in a coupled two ways fashion inspired by Getz et al (34). The first step of the procedure involved clustering genes using a super paramagnetic clustering (SPC) algorithm implemented by Tetko et al (33). This algorithm allows genes to appear in multiple clusters. Similarity between gene expression profiles were measured with Spearman rank correlation. We identified clusters which were stable over a temperature interval of 0.015, and removed overlapping clusters if a they were more than 60% identical and discarding clusters with more than 1000 members. Based on the individual gene clusters we divided the patients into two clusters using hierarchical (agglomerative) clustering with average linkage in Mathematica. Manhattan distance was used to measure similarity between two patient expression profiles. Small patient clusters (3 patients or less) were considered outliers and therefore removed, the remaining patients were reclustered. For visualization the patients were reclustered with hierarchical clustering in cluster by Eisen et al (35). This produced a cluster tree visualized with Treeview (35) of exactly the same clusters as our agglomerative algorithm.

### Defining Transcripts Previously Associated to CAD

Automated text mining of PubMed was used to establish a comprehensive list of genes previously related to CAD and atherosclerosis. Briefly, a gene was considered related if it co-occurred with any of the following terms in the abstract of a published article on Pub Med; coronary artery disease, atherosclerosis and arteriosclerosis. Two other lists were generated manually using cholesterol or diabetes as search terms. Since some established atherosclerosis genes was not captured, some genes were manually extracted from recent CAD and atherosclerosis reviews. The list of CAD-related genes comprised 2832 genes.

### Promoter Analysis

Promoter sequences are from Ensembl v. 43, downloaded from Biomart (http://www.biomart.org/). Transcription factors (TFs) with LIM domain(37)or that are known to interact with LDB2 (38) where identified. From this set of Tfs we searched TRANSFAC v 10.4 (36) for known transcription factor binding sites (TFBS). Seven of theses Tfs had a total of 171 known TFBS. We used the program PATCH (36) and searched for places in the promoter sequences where these 171 motifs match with at least 6 bp without mismatch.

### The Cohorts for Genetic Validation

### SCARF

The Stockholm Coronary Atherosclerosis Risk Factor (SCARF) study is a case-control study, designed to form the basis for studies of genetic and biochemical factors precocious MI. A total of 387 survivors of a first MI aged less than 60 years who had been admitted to the coronary care units of the three hospitals in the northern part of Stockholm (Danderyd Hospital, Karolinska University Hospital Solna and Norrtälje Hospital) were included. Briefly, unselected patients meeting the inclusion criteria were enrolled, and exclusion criteria type 1 diabetes mellitus, renal insufficiency (defined as a plasma creatinine >200 μmol/L), any chronic inflammation disease, drug addiction, psychiatric disease or inability to comply with protocol. For each postinfaretion patient a sex- and age-matched control person was recruited from the general population (response rate 79%). Three months after the index cardiac event, both patients and controls underwent medical examination and blood samples were drawn following an overnight fast. Background data (e.g. social situation, lifestyle, medical history and medication) were collected by means of a structured interviewed. Ethnicity was recorded on the basis of self-reported origin as far as 3 generations back and more than 99% of the participants in the study were considered Caucasians. See also Table 9.

The Stockholm Heart Epidemiology Program (SHEEP) study is a large population-based-case-control study aiming to investigate genetic, biochemical and environmental factors predisposing to MI. Potential study participants (age range 45-70 years) were all Swedish citizens living in Stockholm County without a previous clinical diagnosis of MI. Male cases were recruited between 1992-1994 and female cases between 1992-1994. The criteria for Mi diagnosis were based on guidelines issued the Swedish Society of Cardiology in 1991 and included: (1) typical symptoms; (2) marked elevations of enzymes serum creatine kinase (S-CK) and lactate dehydrogenase (LDH) and (3) characteristic electrocardiogram changes. If two or three criteria were fulfilled, the patient was diagnosed with MI. Five control candidates per case were sampled within two days of the case event, in order to enable replacement of potential non-responders. For each postinfarction patient a randomly selected healthy individual was recruited within two days of the case event, after matching for age, sex and catchment area. Due to a late response from some of the initial controls, occasionally both the initial and the alternative controls have been included. Blood samples were collected approximately three months after the index cardiac event in the patients and all participants underwent physical examination. See also Table 10.

### Results

### Patient Characteristics

The 114 STAGE patients were a typical CAD cohort (Table 5). Importantly, their characteristics did not differ significantly from those of the 66 STAGE patients in whom metabolic gene expression profiles were obtained, who in turn did not differ from the 40 STAGE patients in whom all five expression profiles were obtained. Basic characteristics of the carotid stenosis patients (n=25) from whom gene expression profiles were obtained are also shown in Table 5.

### Gene Expression Related to Extent of Coronary Atherosclerosis

To define gene clusters related to atherosclerosis, we used coupled two-way clustering analysis (Supplementary Methods) on ratios of mRNA from the atherosclerotic aortic root and unaffected IMA for 15,042 refseq transcripts (Methods). Of 14 gene clusters (Table 7, one (n=49 genes) clustered the patients in two groups that differed in the extent of coronary stenosis (P=0.008). Gene clusters identified from liver and skeletal muscle (15 and 11 clusters, respectively; Table 7) did not relate to coronary stenosis. In contrast, two-way clustering of mediastinal fat gene expression profiles generated 20 gene clusters (Table 7); one (n=59) clustered the patients into two groups that differed in extent of coronary stenosis (P=0.00015). Seven genes were present in both atherosclerosis-related clusters (likelihood of occurring by chance (Pc)<7×10⁻¹⁰), indicating common atherosclerosis-related gene activity in mediastinal fat and in the atherosclerotic aortic root.

### Gene Expression Related to Extent of Carotid Atherosclerosis

To validate atherosclerosis-related genes identified in the STAGE cohort, we analyzed a cohort of carotid stenosis patients undergoing carotid surgery (Table 5). Coupled two-way clustering of expression profiles from 25 carotid plaques generated 11 gene clusters (Table 7), one of which (n=55) clustered the patients into two groups that differed in IMT scores (P=0.038). Remarkably, 16 of the 55 genes overlapped with mediastinal fat cluster genes (Pc<9×10⁻²⁷), and 17 with aortic root/IMA cluster genes (Pc<1×10⁻³⁰). Six transcripts (C-type lectin domain family 14, cadherin 5, chromosome 20 open reading frame 160, endothelial differentiation sphingolipid G-protein-coupled receptor-1, G protein-coupled receptor-116, and LDB2) were in all three clusters (Pc<7.15×10⁻²³), and in total there were 129 genes (Table 8).

### Gene Ontology and KEGG Pathway Analyses

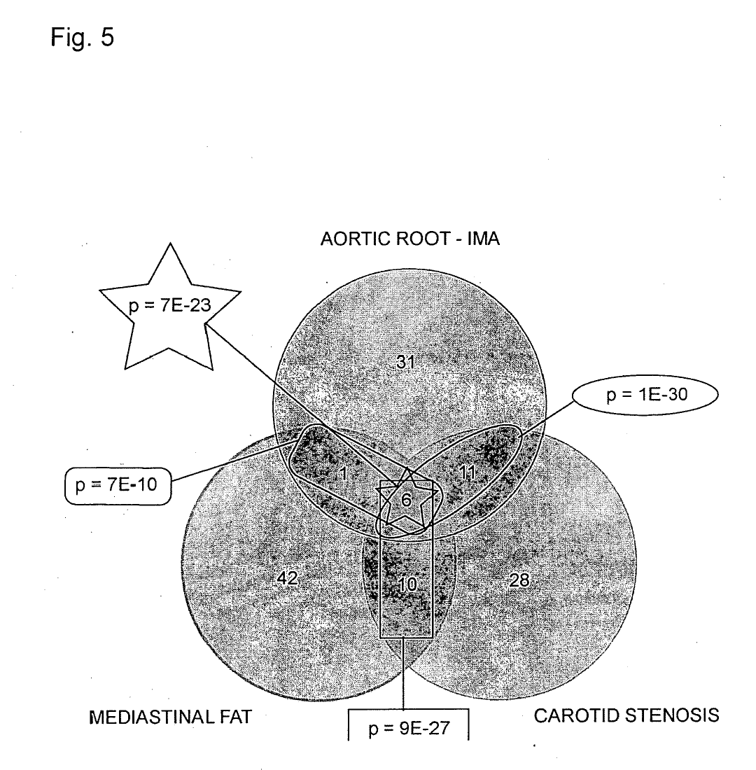

The highly significant overlap between the three identified clusters (relating to the extent of atherosclerosis in three separate tissues from two patient cohorts, FIG. 5) suggested that these clusters harbor biological activity important for atherosclerosis development. To learn more about the genes in these clusters (n=129, Table 8), we performed GO and KEGG pathway analyses. Eighty-nine had a match in the GO category biological process, 100 in molecular functions, and 93 in cellular compartment. The top scores were cell communication (n=40, P<1.4 10⁻⁵), signal transduction (n=37, P=3.7×10⁻⁵), and cell adhesion (n=13, P<6.5×10⁻⁴) in biological process; guanyl-nucleotide exchange factor activity (n=7, P<8.7×10⁻⁵), GTPase regulator activity (n=9, P<2.5×10⁻⁴), and signal transducer activity (n=34, P=3.1×10⁻⁴) in molecular functions; and membrane (n=64, P<1.3 10⁻⁷) and plasma membrane (n=31, P<4.8×10⁻⁷) in cellular compartment. Of the 129 genes (Table 8); 31 genes had previously been related to atherosclerosis, 40 had no biological process annotation and seven were involved in regulatory activity (transcription factors (TFs) and their co-factors). Of the 38 genes that had annotation in KEGG pathways, 16 were associated with the transendothelial migration pathway whereof eight having an exact match (n=8, P<7.7×10⁻⁷).

### Promoter Analysis and Genetic Validation

Of six genes repeatedly represented with higher mRNA levels in relation to the extent of coronary and carotid atherosclerosis (the intersection of all three clusters; FIG. 5), LDB2 stood out as the only regulator of transcription. To explore the possibility that LDB2 could be involved in regulating some of the other 128 genes (the union of all three clusters except for LDB2, FIG. 5), we performed in silico sequence matching for 161 promoters found in 122 of these genes in TRANSFAC. First, we identified seven TFs that have known binding-site motifs in TRANSFAC (v10.4) and LIM-binding domains or other known interaction with LDB2 (ISL-1 alpha, Lmo2, Lhx3a, Lhx3b, LHX2, LHX4, and BRCA1). We found 81% of 161 promoters (target promoters) found in 122 of the 129 genes (94%) that had at least one such binding site. In relation to a background of 10255 human promoters covering [−600, −1] region relative to transcription start sites, binding to the target promoters were statistically enriched by 1.2 to 5 fold.

The cluster and in silico promoter analyses suggested that LDB2 might be relevant for the in vivo regulation of some of the 129 genes related to atherosclerosis severity. If so, functional polymorphisms affecting LDB2 expression should also affect atherosclerosis development. To test this hypothesis, we genetically validated the LDB2 gene in 387 MI survivors with matched controls (39) and in 1091 MI survivors and controls from SHEEP. (24) First we identified five SNPs in LDB2 that were evenly distributed according to LD blocks and then looked for associations with gene expression in the STAGE cohort. Carriers of the minor T allele of SNP rs10939673 tended to have lower LDB2 mRNA levels assessed by real time PCR in the aortic root/IMA (P00.004) and in mediastinal fat (P=0.001). In addition, T allele carriers were significantly underrepresented among MI survivors (P=0.014 (n=375), 0.03 (n=917) and 0.005 (n=1304, combined), Tables 6A-C) and had less atherosclerosis, as judged by coronary stenosis scores (P=0.012, n=375) and plaques area percentage (P=0.029), (Table 6D).

### Summary of Results

Of 60 clusters identified in all tissue types, two related to the extent of coronary stenosis: one in aortic lesions (n=49 genes) and one in mediastinal fat (n=59). Remarkably, 27 of these genes were also identified in a cluster (n=55) relating to extent of atherosclerosis in a validation cohort of carotid stenosis patients. Functional analysis identified transendothelial migration of leukocytes as a common feature of atherosclerosis severity and LIM-domain binding 2 (LDB2) as one out of seven genes related to transcription regulation. In silico promoter analysis suggested that LDB2 indirectly could regulate a large portion of the identified genes. In 387 myocardial infarction survivors with matched controls, the rare T-allele of SNP rs10939673 in LDB2 was underrepresented in survivors and inversely related to coronary atherosclerosis.

### Discussion

Unlike candidate gene or pathway approaches, whole-genome approaches such as global gene expression analysis are more unbiased in relation to prior knowledge of the biological or pathological system under investigation. Thus, whole-genome analyses may rapidly increase our understanding of the molecular mechanisms and common regulators of complex biological problems. In the STAGE study, 15,042 refseq signal values in five CAD-relevant organs were analyzed in each patient to reveal gene activity important for the development of coronary atherosclerosis. One hundred one transcripts in the atherosclerotic aortic wall and in mediastinal visceral fat were related to the extent of coronary atherosclerosis, whereas gene-activity clusters in the liver and skeletal muscle were not. Remarkably, 27 of the 101 transcripts were also found in the only gene-activity cluster related to the extent of atherosclerosis in a validation cohort of 25 carotid stenosis patients.

Bioinformatic evaluation revealed that only 31 of the identified genes had previously been related to atherosclerosis, 40 had no biological process annotation, and 16 were related to the transendothelial leukocyte migration pathway. Of seven transcripts related to transcription regulation, one (LDB2) could potentially regulate up to 81% of the identified transcripts, according to in silico sequence promoter analysis. Genetic validation of LDB2 in two cohorts of MI survivors with population-based controls showed that the minor T-allele of SNP rs10939673 was associated with less coronary stenosis and was significantly less prevalent among MI survivors.

These results suggest that (1) visceral fat in the mediastinum serves as a local source of inflammation affecting coronary atherosclerosis; (2) increased transendothelial migration of leukocytes is associated with greater atherosclerosis severity; (3) LDB2 is a high-hierarchy regulator involved in CAD development; (4) antagonists of LDB2 merit testing as therapies for atherosclerosis, and (5) SNP rs10939673 in LDB2 may be useful in identification of CAD/MI risk.

Transendothelial migration of leukocytes is an established pathway of atherosclerosis development. Monocyte transendothelial migration is essential for foam cell formation and for initiating atherosclerosis plaque development (40, 41) and transendothelial migration of T-cells is thought to be a central process in later phases of atherosclerosis (42). Indeed, transendothelial leukocyte migration has been suggested as a possible target for atherosclerosis treatment. Our KEGG pathway analysis indicated that increased transendothelial migration of leukocytes may be a common feature in patients with more severe atherosclerosis. In addition, some of the identified genes without annotations may have a role in this pathway or its regulation. Moreover, our data suggest that this pathway is involved directly in plaque formation and also indirectly, by increasing the inflammatory status of the mediastinal fat.

No gene clusters related to the degree of coronary stenosis were identified in liver or skeletal muscle. This is surprising considering the importance of these organs for established CAD risk factors such as plasma cholesterol and glucose levels (i.e., diabetes). This finding may reflect normalization of gene expression by therapies for these risk factors. The relation of mediastinal fat (or any visceral fat) to established CAD risk factors in blood is less clear. However, increased hip-waist ratio—an indicator of increased visceral fat mass in the abdomen—is one of the strongest predictors of CAD. Interesting aspects of the mediastinal fat are its anatomic location and recent data suggesting a role of visceral fat as source of inflammatory mediators (43). Although our study does not address how the mediastinal fat may contribute to atherosclerosis, a local source of inflammatory mediators may increase the rate of atherosclerosis progression (44).

Genes encoding LIM domain-binding factors such as LDB2 were initially isolated in a screen for proteins that physically interact with the LIM domains of nuclear proteins. These proteins bind to a variety of TFs and are likely to function as enhancers, bringing together diverse transcription factors to form higher-order activation complexes (45, 46). In our screen of LDB2 associated TFs, ISL-1 alpha, Lmo2, Lhx3a, Lhx3b, Lhx2, Lhx4, and BRCA1 were identified. ISL-1 alpha enhances HNF4 activity and thus insulin signalling (47, 48). Lmo2 is involved in angiogenesis (49, 50). Lhx3 and Lhx4 regulate proliferation and differentiation of pituitary-specific cell lineages (51) and are expressed in subsets of lymphocytes(52) and thymocyte tumor cell lines (53). BRCA1 is associated with a selective deficiency in spontaneous and LPS-induced production of TNF-alpha and of TNF-alpha-induced expression of intercellular adhesion molecule-1 on peripheral blood monocytes (54) and in controlling the life cycle of T lymphocytes (55). Until the current study, LDB2 had not been related to CAD or atherosclerosis. Its high-hierarchy regulatory role and involvement in diverse biological processes make it an interesting target for further evaluation in complex diseases.

In conclusion, molecular profiling of several CAD-relevant organs revealed a distinct molecular atherosclerosis phenotype that was shared by mediastinal fat and replicated in carotid lesions. This phenotype involves transendothelial migration of leukocytes and the TF co-factor LDB2 as a high-hierarchy regulator harboring an atheroprotective rare SNP allele.

### REFERENCES

1. Grines C L. The role of statins in reversing atherosclerosis: What the latest regression studies show. J. Interv. Cardiol. 2006; 19(1):3-9.
2. Tuomisto T T, Binder B R, Yla-Herttuala S. Genetics, genomics and proteomics in atherosclerosis research. Ann Med 2005; 37(5):323-32.
3. Schadt E E, Sachs A, Friend S. Embracing complexity, inching closer to reality. Sci STKE 2005; 2005(295):pe40.
4. Ginsburg G S, Donahue M P, Newby L K. Prospects for personalized cardiovascular medicine: the impact of genomics. J Am Coll Cardiol 2005; 46(9):1615-27.
5. Tegner J, Skogsberg J, Bjorkegren J. Thematic review series: systems biology approaches to metabolic and cardiovascular disorders. Multi-organ whole-genome measurements and reverse engineering to uncover gene networks underlying complex traits. J Lipid Res 2007; 48(2):267-77.
6. Venter J C, Adams M D, Myers E W, Li P W, Mural R J, Sutton G G, et al. The sequence of the human genome. Science 2001; 291(5507):1304-51.
7. Lander E S, Linton L M, Birren B, Nusbaum C, Zody M C, Baldwin J, et al. Initial sequencing and analysis of the human genome. Nature 2001; 409(6822):860-921.
8. Waterston R H, Lindblad-Toh K, Birney E, Rogers J, Abril J F, Agarwal P, et al. Initial sequencing and comparative analysis of the mouse genome. Nature 2002; 420(6915):520-62.
9. Lieu H D, Withycombe S K, Walker Q, Rong J X, Walzem R L, Wong J S, et al. Eliminating Atherogenesis in Mice by Switching Off Hepatic Lipoprotein Secretion. Circulation 2003; 107(9):1315-1321.
10. Tabas I. Consequences and therapeutic implications of macrophage apoptosis in atherosclerosis: the importance of lesion stage and phagocytic efficiency. Arterioseler Thromb Vasc Biol 2005; 25(11):2255-64.
11. Tegner J, Yeung M K, Hasty J, Collins J J. Reverse engineering gene networks: integrating genetic perturbations with dynamical modeling. Proc Natl Acad Sci USA 2003; 100(10):5944-9.
12. Libby P, Geng Y J, Aikawa M, Schoenbeck U, Mach F, Clinton S K, et al. Macrophages and atherosclerotic plaque stability. Curr Opin Lipidol 1996; 7(5):330-5.
13. Schmidt S, Pericak-Vance M A, Sawcer S, Barcellos L F, Hart J, Sims J, et al. Allelic association of sequence variants in the herpes virus entry mediator-B gene (PVRL2) with the severity of multiple sclerosis. 2006; 7(5):384-392.
14. Edqvist J, Blomqvist K. Fusion and fission, the evolution of sterol carrier protein-2. J Mol Evol 2006; 62(3):292-306.
15. Tegner J, Bjorkegren J. Perturbations to uncover gene networks. Trends Genet 2007; 1 (January; 23):34-41.
16. Veniant M M, Sullivan M A, Kim S K, Ambroziak P, Chu A, Wilson M D, et al. Defining the atherogenicity of large and small lipoproteins containing apolipoprotein B100. J Clin Invest 2000; 106(12):1501-10.
17. Stotz E, Schenk E A, Churukian C, Willis C. Oil red O: comparison of staining quality and chemical components as determined by thin layer chromatography. Stain Technol 1986; 61(3):187-90.
18. Basu S K, Goldstein J L, Anderson G W, Brown M S. Degradation of cationized low density lipoprotein and regulation of cholesterol metabolism in homozygous familial hypercholesterolemia fibroblasts. Proc. Natl. Acad. Sci. USA 1976; 73(9):3178-3182.
19. Redgrave T G, Carlson L A. Changes in plasma very low density and low density lipoprotein content, composition, and size after a fatty meal in normo- and hypertriglyceridemic man. J. Lipid Res. 1979; 20(2):217-229.
20. Christoffersen C, Nielsen L B, Axler O, Andersson A, Johnsen A H, Dahlback B. Isolation and characterization of human apolipoprotein M-containing lipoproteins. J. Lipid Res. 2006; 47:1833-11843.
21. Cleveland W. Robust locally weighted regression and smoothing scatterplots. Journal of the American Statistical Association 1979; 74:829-836.
22. Efron B, Tibshirani R, Storey J, Tusher V. Empirical Bayes analysis of a microarray experiment. J. Am. Stat. Assoc. 2001; 96(456):1151-60.
23. Hosack D A, Dennis G, Jr., Sherman B T, Lane H C, Lempicki R A. Identifying biological themes within lists of genes with EASE. Genome Biol 2003; 4(10):R70.
24. Leander K, Hallqvist J, Reuterwall C, Ahlbom A, de Faire U. Family history of coronary heart disease, a strong risk factor for myocardial infarction interacting with other cardiovascular risk factors: results from the Stockholm Heart Epidemiology Program (SHEEP). Epidemiology 2001; 12(2):215-21.
25. Fazio G P, Redberg R F, Winslow T, Schiller N B. Transesophageal echocardiographically detected atherosclerotic aortic plaque is a marker for coronary artery disease. J Am Coll Cardiol 1993; 21(1):144-50.
26. Adler Y, Fisman E Z, Shemesh J, Schwammenthal E, Tanne D, Batavraham I R, et al. Spiral computed tomography evidence of close correlation between coronary and thoracic aorta calcifications. Atherosclerosis 2004; 176(1):133-8.
27. Sims F H. A comparison of coronary and internal mammary arteries and implications of the results in the etiology of arteriosclerosis. Am Heart J 1983; 105(4):560-6.
28. Carlson K. Lipoprotein fractionation. J Clin Pathol Suppl (Assoc Clin Pathol) 1973; 5:32-7.
29. Austen W G, Edwards J E, Frye R L, Gensini G G, Gott V L, Griffith L S, et al. A reporting system on patients evaluated for coronary artery disease. Report of the Ad Hoc Committee for Grading of Coronary Artery Disease, Council on Cardiovascular Surgery, American Heart Association. Circulation 1975; 51(4 Suppl):5-40.
30. Wendelhag I, Liang Q, Gustavsson T, Wikstrand J. A new automated computerized analyzing system simplifies readings and reduces the variability in ultrasound measurement of intima-media thickness. Stroke 1997; 28(11):2195-200.
31. Irizarry R A, Bolstad B M, Collin F, Cope L M, Hobbs B, Speed T P. Summaries of Affymetrix GeneChip probe level data. Nucleic Acids Res 2003; 31(4):e15.
32. Mecham B H, Klus G T, Strovel J, Augustus M, Byrne D, Bozso P, et al. Sequence-matched probes produce increased cross-platform consistency and more reproducible biological results in microarray-based gene expression measurements. Nucleic Acids Res 2004; 32(9):e74.
33. Tetko I V, Facius A, Ruepp A, Mewes H W. Super paramagnetic clustering of protein sequences. BMC Bioinformatics 2005; 6:82.
34. Getz G, Levine E, Domany E. Coupled two-way clustering analysis of gene microarray data. Proc Natl Acad Sci USA 2000; 97(22):12079-84.
35. Eisen M B, Spellman P T, Brown P O, Botstein D. Cluster analysis and display of genome-wide expression patterns. Proc Natl Acad Sci USA 1998; 95(25):14863-8.
36. Matys V, Kel-Margoulis O V, Fricke E, Liebich I, Land S, Barre-Dirrie A, et al. TRANSFAC and its module TRANSCompel: transcriptional gene regulation in eukaryotes. Nucleic Acids Res 2006; 34(Database issue):D108-10.
37. Bateman A, Coin L, Durbin R, Finn R D, Hollich V, Griffiths-Jones S, et al. The Pfam protein families database. Nucleic Acids Res 2004; 32(Database issue):D138-41.
38. von Mering C, Jensen L J, Snel B, Hooper S D, Krupp M, Foglierini M, et al. STRING: known and predicted protein-protein associations, integrated and transferred across organisms. Nucleic Acids Res 2005; 33(Database issue):D433-7.
39. Samnegard A, Silveira A, Lundman P, Boquist S, Odeberg J, Hulthe J, et al. Serum matrix metalloproteinase-3 concentration is influenced by MMP-3-1612 5A/6A promoter genotype and associated with myocardial infarction. J Intern Med 2005; 258(5):411-9.
40. Lusis A J. Atherosclerosis. Nature 2000; 407(6801):233-41.
41. Lusis A J. Genetic factors in cardiovascular disease. 10 questions. Trends Cardiovasc Med 2003; 13(8):309-16.
42. Hansson G K. Inflammation, atherosclerosis, and coronary artery disease. N Engl J Med 2005; 352(16): 1685-95.
43. Berg A H, Scherer P E. Adipose tissue, inflammation, and cardiovascular disease. Circ Res 2005; 96(9):939-49.
44. Mazurek T, Zhang L, Zalewski A, Mannion J D, Diehl J T, Arafat H, et al. Human epicardial adipose tissue Is a source of inflammatory mediators. Circulation 2003; 108(20):2460-2466.
45. Agulnick A D, Taira M, Breen J J, Tanaka T, Dawid I B, Westphal H. Interactions of the LIM-domain-binding factor Ldb1 with LIM homeodomain proteins. Nature 1996; 384(6606):270-2.
46. Jurata L W, Gill G N. Functional analysis of the nuclear LIM domain interactor NLI. Mol Cell Biol 1997; 17(10):5688-98.
47. Kojima H, Nakamura T, Fujita Y, Kishi A, Fujimiya M, Yamada S, et al. Combined expression of pancreatic duodenal homeobox 1 and islet factor 1 induces immature enterocytes to produce insulin. Diabetes 2002; 51(5):1398-408.
48. Eeckhoute J, Briche I, Kurowska M, Formstecher P, Laine B. Hepatocyte nuclear factor 4 alpha ligand binding and F domains mediate interaction and transcriptional synergy with the pancreatic islet LIM HD transcription factor Is11. J Mol Biol 2006; 364(4):567-81.
49. Yamada Y, Pannell R, Forster A, Rabbitts T H. The oncogenic LIM-only transcription factor Lmo2 regulates angiogenesis but not vasculogenesis in mice. Proc Natl Acad Sci USA 2000; 97(1):320-4.
50. Yamada Y, Warren A J, Dobson C, Forster A, Pannell R, Rabbitts T H. The T cell leukemia LIM protein Lmo2 is necessary for adult mouse hematopoiesis. Proc Natl Acad Sci USA 1998; 95(7):3890-5.
51. Sheng H Z, Moriyama K, Yamashita T, Li H, Potter S S, Mahon K A, et al. Multistep control of pituitary organogenesis. Science 1997; 278(5344): 1809-12.
52. Xu Y, Baldassare M, Fisher P, Rathbun G, Oltz E M, Yancopoulos G D, et al. LH-2: a LIM/homeodomain gene expressed in developing lymphocytes and neural cells. Proc Natl Acad Sci USA 1993; 90(1):227-31.
53. Wu H K, Heng H H, Siderovski D P, Dong W F, Okuno Y, Shi X M, et al. Identification of a human LIM-Hox gene, hLH-2, aberrantly expressed in chronic myelogenous leukaemia and located on 9q33-34.1. Oncogene 1996; 12(6):1205-12.
54. Zielinski C C, Budinsky A C, Wagner T M, Wolfram R M, Kostler W J, Kubista M, et al. Defect of tumour necrosis factor-alpha (TNF-alpha) production and TNF-alpha-induced ICAM-1-expression in BRCA1 mutations carriers. Breast Cancer Res Treat 2003; 81(2):99-105.
55. Mak T W, Hakem A, McPherson J P, Shehabeldin A, Zablocki E, Migon E, et al. Brcal required for T cell lineage development but not TCR loci rearrangement. Nat Immunol 2000; 1(1):77-82.
56. Dennis G Jr, Sherman B T, Hosack D A, Yang J, Gao W, Lane H C, Lempicki R A, DAVID: Database for Annotation, Visualization, and Integrated Discovery. Genome Biol. 2003; 4(5):P3

TABLE 1
Plasma cholesterol, triglyceride, and glucose concentrations of representative
Ldlr^{-/-} Apob^{100/100} Mttp^{flox/flox}Mx1-Cre mice

| | Age (weeks) | | | | | |
|---|---|---|---|---|---|---|
| | 10.6 ± 0.4 | 21.3 ± 1.1 | 29.6 ± 0.0 | 40.3 ± 0.0 | 50.5 ± 0.8 | 61.0 ± 0.0 |
| Plasma level (mg/dl) | (n = 7) | (n = 5) | (n = 6) | (n = 5) | (n = 5) | (n = 4) |
| Cholesterol | 361.2 ± 64.2 | 345.7 ± 65.3 | 427.0 ± 72.4 | 461.9 ± 14.1 | 427.2 ± 74.2 | 527.3 ± 71.5 |
| Triglycerides | 83.0 ± 25.8 | 84.1 ± 10.5 | 87.8 ± 17.0 | 84.7 ± 20.0 | 108.6 ± 26.2 | 89.6 ± 38.3 |
| Glucose | 359.6 ± 50.6 | 425.4 ± 76.2 | 369.8 ± 37.8 | 311.4 ± 28.5 | 350.6 ± 19.6 | 376.0 ± 38.3 |

Values are mean ± SD. There were no statistically significant differences between time points (P > 0.05).

TABLE 2
siRNA targeted cholesterol-responsive genes

| | | | Degree of | % CE content | | Visualization of |
|---|---|---|---|---|---|---|
| Gene symbol | | Gene name | knock-down | relative control | p-value^{a} | ORO staining |
| 1. | AGL | amylo-1,6-glucosidase, 4-alpha-glucanotransferase | 63% | −37% | p < 0.001 | decrease |
| 2. | AGPAT3 | 1-acyl-sn-glycerol 3-phosphate acyltransferases | 69% | −14% | ns | decrease |
| 3. | CD36 | CD36 antigen | 66% | −17% | p < 0.001 | degrease |
| 4. | GPR81 | G protein-coupled receptor 81 | 30% | −14% | ns | decrease |
| 5. | GPR120 | G protein-coupled receptor 120 | 67% | +17% | p = 0.04 | increase |
| 6. | GYPC | gypc, glycophorin C | 52% | nd | — | — |
| 7. | HMGB3 | high mobility group box 3 | 65% | −18% | ns | decrease |
| 8. | PRKAR2B | protein kinase, cAMP dependent regulatory, type II beta | 49% | −55% | p = 0.001 | decrease |
| 9. | PVRL2 | poliovirus receptor-related 2 | 65% | −30% | p = 0.002 | decrease |
| 10. | SOX6 | SRY-box containing gene 6 | 72% | −27% | p < 0.001 | decrease |

^{a}P-value indicate CE content in the foam-cells for targeted genes (n = 6) compared with lipofectamine transfected parallel controls (n = 6)
CE indicates cholesterol ester;
ORO, Oil-Red-O;
nd, no difference;
ns, not significant

TABLE 3

| | | | | Smooth | | |
|---|---|---|---|---|---|---|
| | | | | muscle | | |
| | Atherosclerosis- | Macrophage- | Endothelial | cell- | T-cell- | |
| | related | related | cell-related | related | related | Novel |
| Cluster 1 | 36% | 44% | 36% | 25% | 40% | 39% |
| Cluster 2 | 22% | 14% | 15% | 11% | 18% | 66% |
| Cluster 3 | 27% | 22% | 23% | 17% | 26% | 60% |
| Cluster 4 | 20% | 15% | 14% | 12% | 16% | 69% |
| Total | 27% | 24% | 22%% | 16% | 25% | 59% |

TABLE 4
Cholesterol responsive genes in the atherosclerosis wall of 30 weeks old mice

| | | Cholesterol level^{a} | | | |
|---|---|---|---|---|---|
| Gene | | High | Low | Fold | |
| symbol | Gene name | (n = 6) | (n = 7) | change | p-value^{b} |
| Aatk | apoptosis-associated tyrosine | 210 ± 22 | 42 ± 32 | 0.20 | 0.049 |
| | kinase | | | | |
| Agl | amylo-1,6-glucosidase, 4- | 193 ± 28 | 88 ± 31 | 0.45 | 0.050 |
| | alpha-glucanotransferase | | | | |
| Agpat3 | 1-acyl-sn-glycerol 3- | 479 ± 132 | 135 ± 65 | 0.28 | 0.046 |
| | phosphate acyltransferases | | | | |
| Aldh1a1 | aldehyde dehydrogenase | 240 ± 107 | 485 ± 110 | 2.02 | 0.026 |
| | family 1, subfamily A1 | | | | |
| Amy1 | amylase 1, salivary | 1804 ± 642 | 258 ± 187 | 0.14 | 0.0053 |
| Aqp7 | aquaporin 7 | 287 ± 68 | 44.49 ± 35 | 0.15 | 0.0072 |
| Cd36 | CD36 antigen | 2193 ± 944 | 343 ± 222 | 0.16 | 0.0053 |
| Dlat | dihydrolipoamide S- | 2576 ± 834 | 530 ± 356 | 0.21 | 0.040 |
| | acetyltransferase | | | | |
| Etfdh | electron transferring | 4323 ± 2055 | 748 ± 479 | 0.17 | 0.048 |
| | flavoprotein, dehydrogenase | | | | |
| Fmo2 | flavin containing | 343 ± 50 | 574 ± 160 | 1.67 | <0.0001 |
| | monooxygenase 2 | | | | |
| Fndc3b | fibronectin type III domain | 294 ± 41 | 330 ± 50 | 1.13 | 0.035 |
| | containing 3B | | | | |
| Gbe1 | glucan (1,4-alpha-), branching | 717 ± 353 | 108 ± 62 | 0.15 | 0.0061 |
| | enzyme 1 | | | | |
| Gpr81 | G protein-coupled receptor | 278 ± 167 | 36 ± 18 | 0.13 | 0.018 |
| | 81 | | | | |
| Gpr120 | G protein-coupled receptor | 530 ± 231 | 96 ± 64 | 0.18 | 0.023 |
| | 120 | | | | |
| Gprc5b | G protein-coupled receptor, | 548 ± 193 | 90 ± 53 | 0.16 | 0.00074 |
| | family C, group 5, member B | | | | |
| Gypc | gypc, glycophorin C | 520 ± 137 | 188 ± 55 | 0.36 | 0.036 |
| Hmgb3 | high mobility group box 3 | 882 ± 304 | 186 ± 80 | 0.21 | 0.0077 |
| Hsdl2 | hydroxysteroid | 1412 ± 627 | 253 ± 130 | 0.18 | 0.012 |
| | dehydrogenase like 2 | | | | |
| Immt | inner membrane protein, | 663 ± 189 | 176 ± 98 | 0.27 | 0.046 |
| | mitochondrial | | | | |
| Lrpprc | leucine-rich PPR-motif | 501 ± 210 | 117 ± 48 | 0.23 | 0.012 |
| | containing | | | | |
| Mccc2 | methylcrotonoyl-Coenzyme A | 205 ± 34 | 60 ± 40 | 0.29 | 0.038 |
| | carboxylase 2 (beta) | | | | |
| Myl1 | myosin, light polypeptide 1 | 248 ± 108 | 35 ± 24 | 0.14 | 0.0025 |
| Pdhx | pyruvate dehydrogenase | 1147 ± 408 | 223 ± 157 | 0.19 | 0.041 |
| | complex, component X | | | | |
| Ppara | peroxisome proliferator | 428 ± 177 | 55 ± 39 | 0.13 | 0.030 |
| | activated receptor alpha | | | | |
| Prkar2b | protein kinase, cAMP | 1819 ± 731 | 343 ± 222 | 0.15 | 0.040 |
| | dependent regulatory, type II | | | | |
| | beta | | | | |
| Pvrl2 | poliovirus receptor-related 2 | 220 ± 55 | 62 ± 23 | 0.28 | 0.0033 |
| Slc16a1 | solute carrier family 16 | 286 ± 129 | 55 ± 30 | 0.19 | 0.017 |
| | (monocarboxylic acid | | | | |
| | transporters), member 1 | | | | |
| Sox6 | SRY-box containing gene 6 | 174 ± 42 | 65 ± 18 | 0.38 | 0.0035 |
| Stat1 | signal transducer and activator | 374 ± 154 | 115 ± 36 | 0.31 | 0.049 |
| | of transcription 1 | | | | |
| 2210412D01 | RIKEN cDNA 2210412D01 | 247 ± 84 | 81 ± 28 | 0.33 | 0.042 |
| Rik | gene | | | | |
| 2310042G06 | RIKEN cDNA 2310042G06 | 1284 ± 316 | 387 ± 142 | 0.30 | 0.037 |
| Rik | gene | | | | |
| 2310079P10 | RIKEN cDNA 2310079P10 | 370 ± 78 | 55 ± 30 | 0.14 | 0.0027 |
| Rik | gene | | | | |
| 2610002J23 | RIKEN cDNA 2610002J23 | 188 ± 51 | 292 ± 66 | 1.56 | 0.048 |
| Rik | gene | | | | |
| 4933429D07 | RIKEN cDNA 4933429D07 | 598 ± 247 | 68 ± 51 | 0.12 | 0.0017 |
| Rik | gene | | | | |
| AA408954 | expressed sequence AA408954 | 2576 ± 834 | 530 ± 356 | 0.21 | 0.040 |
| BC034068 | cDNA sequence BC034068 | 1518 ± 595 | 172 ± 137 | 0.11 | 0.012 |
| D0H4S114 | DNA segment, human D4S114 | 269 ± 68 | 546 ± 199 | 2.03 | 0.037 |
| D830041H16 | RIKEN cDNA D830041H16 | 663 ± 189 | 176 ± 98 | 0.27 | 0.046 |
| Rik | gene | | | | |

TABLE 5
Basic characteristics of the patients in the STAGE study

| | | STAGE | STAGE | |
|---|---|---|---|---|
| | | metabolic | complete | Carotid lesion |
| | STAGE | expression | expression | expression |
| | cohort | profiles | profiles | profiles |
| Characteristics | (N = 114) | (N = 66) | P (N = 40) | P (N = 25) |
| Continuous variables | | | | |
| Mean ± SD | | | | |
| Age - yr | 66 ± 8 | 66 ± 8 | 66 ± 8 | 69 ± 11 |
| Body-mass index | 26.6 ± 3.7 | 26.4 ± 3.9 | 26.3 ± 3.9 | 25.3 ± 3.2 |
| Waist-to-hip ratio | 0.94 ± 0.06 | 0.93 ± 0.06 | 0.93 ± 0.06 | 0.91 ± 0.07 |
| Blood pressure - mmHg | | | | |
| Systolic | 141 ± 19 | 140 ± 19 | 135 ± 18 | 150 ± 19 |
| Diastolic | 80 ± 9 | 80 ± 10 | 78 ± 8 | 77 ± 9 |
| Insulin - pmol/L | 62 ± 47 | 59 ± 49 | 61 ± 53 | 44 ± 16 |
| Proinsulin - pmol/L | 5.6 ± 5.7 | 5.1 ± 5.7 | 5.5 ± 6.9 | 4.6 ± 2.4 |
| Cholesterol - mmol/L | | | | |
| Total | 4.08 ± 1.01 | 3.97 ± 1.08 | 3.83 ± 1.02 | 4.74 ± 1.21 |
| VLDL | 0.32 ± 0.25 | 0.29 ± 0.25 | 0.26 ± 0.25 | 0.22 ± 0.17 |
| LDL | 2.09 ± 0.79 | 2.01 ± 0.84 | 1.87 ± 0.76 | 2.60 ± 0.90 |
| HDL | 1.49 ± 0.29 | 1.51 ± 0.33 | 1.54 ± 0.39 | 1.74 ± 0.48 |
| Triglycerides - mmol/L | | | | |
| Total | 1.41 ± 0.73 | 1.36 ± 0.70 | 1.41 ± 0.76 | 1.23 ± 0.49 |
| VLDL | 1.04 ± 0.67 | 0.97 ± 0.64 | 0.98 ± 0.68 | 0.79 ± 0.42 |
| LDL | 0.26 ± 0.09 | 0.27 ± 0.09 | 0.28 ± 0.09 | 0.29 ± 0.09 |
| HDL | 0.16 ± 0.05 | 0.17 ± 0.05 | 0.19 ± 0.06** | 0.20 ± 0.08 |
| Alcohol consumption - g/week | 120 ± 96 | 117 ± 89 | 124 ± 82 | 117 ± 106 |
| Atherosclerotic score | | | | |
| Plaque area¥ | NA | 37.8 ± 14.1 | 38.3 ± 13.8 | NA |
| Stenosis score | NA | 5.06 ± 2.41 | 5.37 ± 2.43 | NA |
| IMT | NA | NA | NA | 1.24 ± 0.24 |
| Rehabilitation days | NA | 13 ± 8 | 13 ± 7 | NA |

Categorical variables no. of subjects (%)

| | | | | |
|---|---|---|---|---|
| Sex | | | | |
| Male | 102 (89) | 59 (89) | 37 (93) | 15 (60) |
| Female | 12 (11) | 7 (11) | 3 (8) | 10 (40) |
| Smokers | | | | |
| Current | 8 (7) | 4 (6) | 2 (5) | 1 (4) |
| Former | 70 (61) | 42 (64) | 25 (63) | 18 (67) |
| Non | 36 (32) | 20 (30) | 13 (33) | 5 (21) |
| Present disease | | | | |
| Diabetes | 24 (21) | 11 (17) | 5 (13)* | 2 (8) |
| Hypertony | 72 (63) | 43 (65) | 25 (63) | 16 (64) |
| Hyperlipidemia | 84 (74) | 49 (74) | 27 (68) | 13 (52) |
| Treatment | | | | |
| Statins | 101 (89) | 61 (92) | 37 (93) | 15 (60) |
| Betablocker | 103 (90) | 62 (94) | 38 (95) | 11 (44) |
| Insulin (oral or subcutaneous) | 23 (20) | 9 (14) | 5 (13) | 1 (4) |
| Postoperative complications | | | | |
| Major cardiovascular event | NA | 9 (14) | 6 (15) | NA |
| Minor cardiovascular event | NA | 13 (20) | 8 (20) | NA |

P-values are calculated using unpaired t-test comparing subgroups in STAGE with the entire STAGE cohort (N = 114).
*P-value <0.05;
**P-value <0.01
¥Plaque area is summarized over seven segments in the left coronary artery.
Numbers in parenthesis show % of whole subgroup

TABLE 6
Association analyses of rs10939673 in two cohorts of MI cases and matched controls
(a) Genotype and allele frequencies for rs10939673 in 375 MI and controls

| | Genotype distribution | | | | Allele frequencies | | | | T dominant model | | | |
|---|---|---|---|---|---|---|---|---|---|---|---|---|
| | CC | CT | TT | p-value | C | T | MAF | p-value | CC | CT + TT | OR (95% c.i.) | p-value |
| MI patients | 160 | 170 | 45 | 0.047 | 490 | 260 | 0.347 | 0.034 | 160 | 215 | 0.69 (0.51-0.93) | 0.014 |
| MI controls | 130 | 200 | 53 | | 460 | 306 | 0.399 | | 130 | 253 | | |

(b) Genotype and allele frequencies for rs10939673 in SHEEP

| | |
|---|---|
| Allele frequencies | T dominant model |

| | C | T | MAF | p-value | CC | CT + TT | OR (95% c.i.) | p-value |
|---|---|---|---|---|---|---|---|---|
| MI patients | 1142 | 716 | 0.385 | 0.031 | 351 | 578 | 0.86 (−∞-0.996) | 0.046 |
| MI controls | 1506 | 1060 | 0.413 | | 440 | 843 | | |

(c) Genotype and allele frequencies for rs10939673 in both MI cohorts

| | Genotype distribution | | | | Allele frequencies | | | | T dominant model | | | |
|---|---|---|---|---|---|---|---|---|---|---|---|---|
| | CC | CT | TT | p-value | C | T | MAF | p-value | CC | CT + TT | OR (95% c.i.) | p-value |
| MI patients | 511 | 610 | 183 | 0.015 | 1632 | 976 | 0.374 | 0.005 | 511 | 793 | 0.81 (0.69-0.94) | 0.005 |
| MI controls | 570 | 826 | 270 | | 1966 | 1366 | 0.410 | | 570 | 1096 | | |

(d) Associations between ra10939673 and atherosclerosis measurements in 375 MI cases

| | |
|---|---|
| Genotype - Patients | Genotype - Patients |

| Variable | CC | CT | TT | p-value | CC | CT + TT | p-value |
|---|---|---|---|---|---|---|---|
| Stenosis score | 8.7 ± 2.9 | 7.7 ± 3.0 | 7.5 ± 3.9 | 0.040 | 8.7 ± 2.9 | 7.6 ± 3.2 | 0.012 |
| Plaque area | 68 ± 30 | 62 ± 20 | 53 ± 27 | 0.024 | 68 ± 30 | 60 ± 22 | 0.029 |

MAF indicates minor allele frequency;
MI, myocardial infarction
Plaque area is summarized over all segments.

TABLE 7
Coupled two-way gene clusters associated to
stenosis score or IMT in all tissues.

| | Stenosis Score | | | | | |
|---|---|---|---|---|---|---|
| | Mean #1 | Mean #2 | P-value | FDR | N 1 | N 2 |
| Aortic | | | | | | |
| root-IMA | | | | | | |
| Cluster 1 | 5.8125 | 8.2 | — | — | 32 | 5 |
| Cluster 2 | 5.071429 | 6.72 | 0.040 | 0.281 | 14 | 25 |
| Cluster 3 | 6.363636 | 4.833333 | — | — | 33 | 6 |
| Cluster 4 | 6.7 | 5.0625 | 0.060 | 0.290 | 20 | 16 |
| Cluster 5 | 4.875 | 7 | 0.008 | 0.100 | 16 | 23 |
| Cluster 6 | 6.583333 | 5.384615 | 0.172 | 0.535 | 24 | 13 |
| Cluster 7 | 6.060606 | 6.5 | — | — | 33 | 6 |
| Cluster 8 | 5.558824 | 10 | — | — | 34 | 5 |
| Cluster 9 | 6.269231 | 6.25 | — | — | 26 | 4 |
| Cluster 10 | 5.3125 | 9.857143 | — | — | 32 | 7 |
| Cluster 11 | 4 | 6.266667 | — | — | 5 | 30 |
| Cluster 12 | 6.241379 | 5.555556 | 0.553 | 0.939 | 29 | 9 |
| Cluster 13 | 6.058824 | 6.6 | — | — | 34 | 5 |
| Cluster 14 | 5.266667 | 6.73913 | 0.083 | 0.330 | 15 | 23 |
| Liver | | | | | | |
| Cluster 1 | 5.612903 | 3.666667 | — | — | 62 | 3 |
| Cluster 2 | 5.666667 | 5.333333 | 0.652 | 0.939 | 42 | 21 |
| Cluster 3 | 5.545455 | 5.4 | 0.844 | 0.963 | 55 | 10 |
| Cluster 4 | 5.604167 | 5.294118 | 0.676 | 0.939 | 48 | 17 |
| Cluster 5 | 5.534483 | 5.5 | — | — | 58 | 4 |
| Cluster 6 | 5.54902 | 5.454545 | 0.937 | 0.963 | 51 | 11 |
| Cluster 7 | 5.647059 | 5.5 | 0.837 | 0.963 | 17 | 44 |
| Cluster 8 | 5.551724 | 5.285714 | — | — | 58 | 7 |
| Cluster 9 | 5.551724 | 5.285714 | — | — | 58 | 7 |
| Cluster 10 | 5.466667 | 5.575758 | 0.875 | 0.963 | 30 | 33 |
| Cluster 11 | 5.571429 | 5.222222 | 0.668 | 0.939 | 56 | 9 |
| Cluster 12 | 5.403509 | 5 | — | — | 57 | 5 |
| Cluster 13 | 5.525424 | 5.5 | — | — | 59 | 4 |
| Cluster 14 | 5.411765 | 6.090909 | 0.568 | 0.939 | 51 | 11 |
| Cluster 15 | 5.6 | 5.1 | 0.493 | 0.911 | 55 | 10 |
| Skeletal muscle | | | | | | |
| Cluster 1 | 5.871795 | 5 | 0.263 | 0.663 | 39 | 21 |
| Cluster 2 | 5.74359 | 5.04 | 0.318 | 0.718 | 39 | 25 |
| Cluster 3 | 5.666667 | 5.4 | 0.717 | 0.953 | 36 | 25 |
| Cluster 4 | 5.509434 | 5.625 | 0.893 | 0.963 | 53 | 8 |
| Cluster 5 | 5.680851 | 4.785714 | 0.297 | 0.713 | 47 | 14 |
| Cluster 6 | 4.967742 | 6 | 0.128 | 0.438 | 31 | 32 |
| Cluster 7 | 5.466667 | 6.2 | — | — | 60 | 5 |
| Cluster 8 | 5.509091 | 5.6 | 0.943 | 0.963 | 55 | 10 |
| Cluster 9 | 5.509091 | 5.714286 | — | — | 55 | 7 |
| Cluster 10 | 5.535714 | 5 | 0.685 | 0.939 | 56 | 8 |
| Cluster 11 | 5.509434 | 5.625 | 0.893 | 0.963 | 53 | 8 |
| Mediastinal fat | | | | | | |
| Cluster 1 | 5.533333 | 5.5 | — | — | 60 | 4 |
| Cluster 2 | 5.679245 | 4.833333 | 0.394 | 0.822 | 53 | 12 |
| Cluster 3 | 5.958333 | 4.294118 | 0.044 | 0.281 | 48 | 17 |
| Cluster 4 | 5.045455 | 6.52381 | 0.047 | 0.281 | 44 | 21 |
| Cluster 5 | 3.842105 | 6.217391 | 0.000 | 0.007 | 19 | 46 |
| Cluster 6 | 5.414634 | 5.772727 | 0.644 | 0.939 | 41 | 22 |
| Cluster 7 | 5.517857 | 5.555556 | 0.960 | 1.000 | 56 | 9 |
| Cluster 8 | 5.98 | 4 | 0.004 | 0.057 | 50 | 15 |
| Cluster 9 | 5.177778 | 6.3 | 0.125 | 0.438 | 45 | 20 |
| Cluster 10 | 5.724138 | 3.857143 | — | — | 58 | 7 |
| Cluster 11 | 5.784314 | 4.571429 | 0.199 | 0.535 | 51 | 14 |
| Cluster 12 | 5.784314 | 4.571429 | 0.199 | 0.535 | 51 | 14 |
| Cluster 13 | 5.942857 | 5.071429 | 0.201 | 0.535 | 35 | 28 |
| Cluster 14 | 5.958333 | 4.294118 | 0.044 | 0.281 | 48 | 17 |
| Cluster 15 | 5.418182 | 6.1 | 0.444 | 0.888 | 55 | 10 |
| Cluster 16 | 5.461538 | 5.333333 | 0.862 | 0.963 | 52 | 12 |
| Cluster 17 | 5.425926 | 6 | 0.487 | 0.911 | 54 | 11 |
| Cluster 18 | 5.483333 | 6 | — | — | 60 | 5 |
| Cluster 19 | 5.333333 | 6.214286 | 0.329 | 0.718 | 51 | 14 |
| Cluster 20 | 5.452381 | 5.380952 | 0.916 | 0.963 | 42 | 21 |

| | IMT value | | | | |
|---|---|---|---|---|---|
| Carotid stenosis | Mean #1 | Mean #2 | P-value | N 1 | N 2 |
| Cluster 1 | 1.227462 | 1.263667 | 0.741422 | 14 | 10 |
| Cluster 2 | 1.219 | 1.3214 | — | 18 | 6 |
| Cluster 3 | 1.192765 | 1.4106 | — | 18 | 6 |
| Cluster 4 | 1.237765 | 1.339333 | — | 18 | 4 |
| Cluster 5 | 1.193294 | 1.4088 | — | 19 | 5 |
| Cluster 6 | 1.2932 | 1.211818 | 0.469841 | 10 | 13 |
| Cluster 7 | 1.136 | 1.354636 | 0.039084 | 10 | 13 |
| Cluster 8 | 1.226067 | 1.2715 | — | 16 | 5 |
| Cluster 9 | 1.197 | 1.2321 | 0.733146 | 8 | 11 |
| Cluster 10 | 1.235077 | 1.286286 | 0.631093 | 14 | 8 |
| Cluster 11 | 1.261 | 1.202143 | — | 17 | 7 |

Patient groups <8 were not considered.

TABLE 8
129 genes in the union of the three identified clusters in aorta - IMA,
mediastinal fat and carotid lesion.

| | Gene | |
|---|---|---|
| RefSeq | Symbol | Gene name |
| NM_000054 | AVPR2 | arginine vasopressin receptor 2 (nephrogenic diabetes insipidus) |
| NM_000148 | FUT1 | fucosyltransferase 1 (galactoside 2-alpha-L-fucosyltransferase, |
| | | H blood group) |
| NM_000334 | SCN4A | sodium channel, voltage-gated, type IV, alpha |
| NM_000355 | TCN2 | transcobalamin II; macrocytic anemia |
| NM_000442 | PECAM1 | platelet/endothelial cell adhesion molecule (CD31 antigen) |
| NM_000459 | TEK | TEK tyrosine kinase, endothelial (venous malformations, |
| | | multiple cutaneous and mucosal) |
| NM_000552 | VWF | von Willebrand factor |
| NM_000609 | CXCL12 | chemokine (C—X—C motif) ligand 12 (stromal cell-derived |
| | | factor 1) |
| NM_000681 | ADRA2A | adrenergic, alpha-2A-, receptor |
| NM_000717 | CA4 | carbonic anhydrase IV |
| NM_001010846 | SHE | Src homology 2 domain containing E |
| NM_001046 | SLC12A2 | solute carrier family 12 (sodium/potassium/chloride |
| | | transporters), member 2 |
| NM_001084 | PLOD3 | procollagen-lysine, 2-oxoglutarate 5-dioxygenase 3 |
| NM_001289 | CLIC2 | chloride intracellular channel 2 |
| NM_001290 | LDB2 | LIM domain binding 2 |
| NM_001312 | CRIP2 | cysteine-rich protein 2 |
| NM_001400 | EDG1 | endothelial differentiation, sphingolipid G-protein-coupled |
| | | receptor, 1 |
| NM_001552 | IGFBP4 | insulin-like growth factor binding protein 4 |
| NM_001718 | BMP6 | bone morphogenetic protein 6 |
| NM_001795 | CDH5 | cadherin 5, type 2, VE-cadherin (vascular epithehum) |
| NM_001882 | CRHBP | corticotropin releasing hormone binding protein |
| NM_001889 | CRYZ | crystallin, zeta (quinone reductase) |
| NM_002017 | FLI1 | Friend leukemia virus integration 1 |
| NM_002060 | GJA4 | gap junction protein, alpha 4, 37 kDa (connexin 37) |
| NM_002073 | GNAZ | guanine nucleotide binding protein (G protein), alpha z |
| | | polypeptide |
| NM_002253 | KDR | kinase insert domain receptor (a type III receptor tyrosine |
| | | kinase) |
| NM_002290 | LAMA4 | laminin, alpha 4 |
| NM_002343 | LTF | lactotransferrin |
| NM_002599 | PDE2A | phosphodiesterase 2A, cGMP-stimulated |
| NM_002661 | PLCG2 | phospholipase C, gamma 2 (phosphatidylinositol-specific) |
| NM_002837 | PTPRB | protein tyrosine phosphatase, receptor type, B |
| NM_002856 | PVRL2 | poliovirus receptor-related 2 (herpesvirus entry mediator B) |
| NM_003265 | TLR3 | toll-like receptor 3 |
| NM_003277 | CLDN5 | claudin 5 (transmembrane protein deleted in velocardiofacial |
| | | syndrome) |
| NM_003810 | TNFSF10 | tumor necrosis factor (ligand) superfamily, member 10 |
| NM_004444 | EPHB4 | EPH receptor B4 |
| NM_004510 | SP110 | sp110 nuclear body protein |
| NM_004557 | NOTCH4 | Notch homolog 4 (Drosophila) |
| NM_004955 | SLC29A1 | solute carrier family 29 (nucleoside transporters), member 1 |
| NM_005161 | AGTRL1 | angiotensin II receptor-like 1 |
| NM_005424 | TIE1 | tyrosine kinase with immunoglobulin-like and EGF-like |
| | | domains 1 |
| NM_005512 | LRRC32 | leucine rich repeat containing 32 |
| NM_005693 | NR1H3 | nuclear receptor subfamily 1, group H, member 3 |
| NM_005723 | TSPAN5 | tetraspanin 5 |
| NM_005795 | CALCRL | calcitonin receptor-like |
| NM_005797 | EVA1 | epithelial V-like antigen 1 |
| NM_005856 | RAMP3 | receptor (calcitonin) activity modifying protein 3 |
| NM_006105 | RAPGEF3 | Rap guanine nucleotide exchange factor (GEF) 3 |
| NM_006255 | PRKCH | protein kinase C, eta |
| NM_006334 | OLFM1 | olfactomedin 1 |
| NM_006435 | IFITM2 | interferon induced transmembrane protein 2 (1-8D) |
| NM_007023 | RAPGEF4 | Rap guanine nucleotide exchange factor (GEF) 4 |
| NM_007177 | FAM107A | family with sequence similarity 107, member A |
| NM_012072 | C1QR1 | complement component 1, q subcomponent, receptor 1 |
| NM_012307 | EPB41L3 | erythrocyte membrane protein band 4.1-like 3 |
| NM_013355 | PKN3 | protein kinase N3 |
| NM_014220 | TM4SF1 | transmembrane 4 L six family member 1 |
| NM_014421 | DKK2 | dickkopf homolog 2 (Xenopus laevis) |
| NM_014550 | CARD10 | caspase recruitment domain family, member 10 |
| NM_014725 | STARD8 | START domain containing 8 |
| NM_014839 | LPPR4 | plasticity related gene 1 |
| NM_015234 | GPR116 | G protein-coupled receptor 116 |
| NM_015460 | MYRIP | myosin VIIA and Rab interacting protein |
| NM_015568 | PPP1R16B | protein phosphatase 1, regulatory (inhibitor) subunit 16B |
| NM_015660 | GIMAP2 | GTPase, IMAP family member 2 |
| NM_015937 | PIGT | phosphatidylinositol glycan, class T |
| NM_016242 | EMCN | endomucin |
| NM_016511 | CLEC1A | C-type lectin domain family 1, member A |
| NM_016580 | PCDH12 | protocadherin 12 |
| NM_016929 | CLIC5 | chloride intracellular channel 5 |
| NM_017577 | GRAMD1C | GRAM domain containing 1C |
| NM_017719 | SNRK | SNF related kinase |
| NM_017805 | RASIP1 | Ras interacting protein 1 |
| NM_018058 | CRTAC1 | cartilage acidic protein 1 |
| NM_018326 | GIMAP4 | GTPase, IMAP family member 4 |
| NM_018664 | SNFT | jun dimerization protein p21snft |
| NM_018728 | MYO5C | myosin VC |
| NM_019055 | ROBO4 | roundabout homolog 4, magic roundabout (Drosophila) |
| NM_019555 | ARHGEF3 | Rho guanine nucleotide exchange factor (GEF) 3 |
| NM_020130 | C8orf4 | chromosome 8 open reading frame 4 |
| NM_020142 | NDUFA4L2 | nadh:ubiquinone oxidoreductase mlrq subunit homolog |
| NM_020766 | PCDH19 | protocadherin 19 |
| NM_020812 | DOCK6 | dedicator of cytokinesis 6 |
| NM_021034 | IFITM3 | interferon induced transmembrane protein 3 (1-8U) |
| NM_022003 | FXYD6 | FXYD domain containing ion transport regulator 6 |
| NM_022164 | TINAGL1 | tubulointerstitial nephritis antigen-like 1 |
| NM_022454 | SOX17 | SRY (sex determining region Y)-box 17 |
| NM_022481 | CENTD3 | centaurin, delta 3 |
| NM_022770 | NM_022770 | hypothetical protein flj13912 |
| NM_022821 | ELOVL1 | elongation of very long chain fatty acids (FEN1/Elo2, |
| | | SUR4/Elo3, yeast)-like 1 |
| NM_024689 | CXorf36 | chromosome X open reading frame 36 |
| NM_024756 | MMRN2 | multimerin 2 |
| NM_024781 | C18orf14 | chromosome 18 open reading frame 14 |
| NM_024869 | GRRP1 | glycine/arginine rich protein 1 |
| NM_025179 | PLXNA2 | plexin A2 |
| NM_031310 | PLVAP | plasmalemma vesicle associated protein |
| NM_031439 | SOX7 | SRY (sex determining region Y)-box 7 |
| NM_052946 | NOSTRIN | nitric oxide synthase trafficker |
| NM_052954 | CYYR1 | cysteine/tyrosine-rich 1 |
| NM_052960 | RBP7 | retinol binding protein 7, cellular |
| NM_052970 | HSPA12B | heat shock 70 kD protein 12B |
| NM_080388 | S100A16 | S100 calcium binding protein A16 |
| NM_080625 | C20orf160 | chromosome 20 open reading frame 160 |
| NM_133625 | SYN2 | synapsin ii |
| NM_138786 | TM4SF18 | transmembrane 4 L six family member 18 |
| NM_138961 | ESAM | endothelial cell adhesion molecule |
| NM_139247 | ADCY4 | adenylate cyclase 4 |
| NM_144649 | TMEM71 | transmembrane protein 71 |
| NM_144765 | EVA1 | epithelial v-like antigen 1 |
| NM_145057 | CDC42EP5 | CDC42 effector protein (Rho GTPase binding) 5 |
| NM_145273 | CD300LG | CD300 antigen like family member G |
| NM_152354 | ZNF285 | zinc finger protein 285 |
| NM_152400 | NM_152400 | hypothetical protein flj39370 |
| NM_152406 | NM_152406 | hypothetical protein flj36748 |
| NM_152536 | FGD5 | FYVE, RhoGEF and PH domain containing 5 |
| NM_152727 | CPNE2 | copine II |
| NM_170600 | SH2D3C | sh2 domain containing 3c |
| NM_173505 | ANKRD29 | ankyrin repeat domain 29 |
| NM_173728 | ARHGEF15 | Rho guanine nucleotide exchange factor (GEF) 15 |
| NM_174934 | SCN4B | sodium channel, voltage-gated, type IV, beta |
| NM_174938 | FRMD3 | FERM domain containing 3 |
| NM_175060 | CLEC14A | C-type lectin domain family 14, member A |
| NM_175571 | GIMAP8 | GTPase, IMAP family member 8 |
| NM_175744 | RHOC | ras homolog gene family, member C |
| NM_178172 | LOC338328 | high density lipoprotein-binding protein |
| NM_181844 | BCL6B | B-cell CLL/lymphoma 6, member B (zinc finger protein) |
| NM_182487 | OLFML2A | olfactomedin-like 2A |
| NM_198471 | ANKRD47 | ankyrin repeat domain 47 |
| NM_199054 | MKNK2 | map kinase interacting serine/threonine kinase 2 |

TABLE 9
Basic characteristics of the 387 MI patients and control subjects

| | | Controls | |
|---|---|---|---|
| | Patients (n = 387) | (n = 387) | P-value |
| Age | 54 (49-57) | 54 (49-57) | |
| Male (%) | 82 | 82 | |
| Smokers (%) | 50 | 25 | <0.0001 |
| Family history of | 42 | 21 | <0.0001 |
| CHD (%) | | | |
| Diabetes (%) | 11 | 0 | <0.0001 |
| Hypertension (%) | 34 | 6 | <0.0001 |
| Hyperlipidaemia (%) | 70 | 16 | <0.0001 |
| BMI (kg m⁻²) | 26.8 (24.7-29.7) | 25.6 (23.8-27.8) | <0.0001 |
| SBP (mmHg) | 130 (118-140) | 128 (118-140) | ns |
| DBP (mmHg) | 80 (75-88) | 80 (78-88) | ns |
| Glucose (mmol L⁻¹) | 5.3 (5.0-5.9) | 4.8 (4.6-5.2) | <0.0001 |
| Total cholesterol | 5.0 (4.3-5.7) | 5.4 (4.7-6.1) | <0.0001 |
| (mmol L⁻¹) | | | |
| Triglycerides (mmol L⁻¹) | 1.6 (1.2-2.2) | 1.2 (0.8-1.6) | <0.0001 |
| LDL cholesterol | 3.2 (2.5-3.9) | 3.4 (2.9-4.2) | <0.0001 |
| (mmol L⁻¹) | | | |
| HDL cholesterol | 1.1 (0.9-1.3) | 1.4 (1.1-1.6) | <0.0001 |
| (mmol L⁻¹) | | | |
| Insulin (pmol L⁻¹) | 47.0 (32.0-69.0) | 36.0 (27.5-50.5) | <0.0001 |
| Proinsulin (pmol L⁻¹) | 5.1 (3.4-7.5) | 3.5 (2.6-5.4) | <0.0001 |
| CRP (mg L⁻¹) | 1.5 (0.7-3.4) | 1.0 (0.5-1.8) | <0.0001 |
| IL-6 (pg mL⁻¹) | 0.8 (0.6-1.4) | 0.6 (0.5-1.0) | <0.0001 |
| Cystatin C (mg L⁻¹) | 0.81 (0.73-0.93) | 0.83 (0.77-0.90) | ns |
| Fibrinogen (g L⁻¹) | 3.8 (3.3-4.4) | 3.5 (3.1-4.0) | <0.0001 |
| PAI-1 (IU mL⁻¹) | 12.6 (4.8-22.8) | 7.5 (3.0-17.6) | <0.0001 |

Values expressed are median (interquartile range) or percentage.
CHD, coronary heart disease;
BMI, body mass index;
SBP, systolic blood pressure;
DBP, diastolic blood pressure;
LDL, low-density lipoprotein;
HDL, high-density lipoprotein;
CRP, C-reactive protein;
IL-6, interleukin-6;
PAI-1, plasminogen activator inhibitor-1.

TABLE 10
General characteristics and genotype frequency distributions according to gender and case-control status

| | Men | | | | | Women | | | | |
|---|---|---|---|---|---|---|---|---|---|---|
| Variable | Cases | n | Controls | n | P-value | Cases | n | Controls | n | P-value |
| Age (years) | 58.3 (7.1) | 852 | 58.8 (7.1) | 1054 | — | 61.6 (6.8) | 361 | 62.0 (6.7) | 507 | — |
| Physical inactivity (%) | 41.0 | 334 | 32.3 | 331 | <0.001 | 54.8 | 189 | 41.2 | 205 | <0.001 |

Smoking (%)

| | | | | | | | | | | |
|---|---|---|---|---|---|---|---|---|---|---|
| Never (%) | 20.5 | 169 | 35.5 | 369 | <0.001 | 35.2 | 122 | 51.3 | 257 | <0.001 |
| Former and current (%) | 79.5 | 656 | 64.5 | 669 | | 64.8 | 225 | 48.7 | 244 | |
| Waist-to-hip ratio | 0.96 (0.06) | 847 | 0.94 (0.06) | 1044 | <0.001 | 0.86 (0.09) | 361 | 0.84 (0.09) | 503 | <0.001 |
| Hypertension (%)^{a} | 44.8 | 382 | 50.9 | 537 | 0.008 | 49.6 | 179 | 55.2 | 280 | 0.10 |
| Hypercholesterolemia (%)^{b} | 83.0 | 705 | 78.2 | 823 | 0.008 | 89.1 | 320 | 84.6 | 427 | 0.05 |
| Triglycerides (mmol L⁻¹) | 1.8 (1.7-1.8) | 850 | 1.4 (1.3-1.4) | 1053 | <0.001 | 1.8 (1.7-1.8) | 359 | 1.2 (1.2-1.3) | 505 | <0.001 |
| Insulin (μU mL⁻¹) | 11.0 (10.4-11.6) | 602 | 8.8 (8.5-9.2) | 754 | <0.001 | 11.1 (10.3-12.0) | 297 | 8.3 (7.8-8.7) | 414 | <0.001 |
| Fibrinogen (g L⁻¹) | 3.6 (3.5-3.6) | 789 | 3.4 (3.3-3.4) | 992 | <0.001 | 3.8 (3.7-3.9) | 341 | 3.5 (3.5-3.6) | 467 | <0.001 |
| IL-6 (ng L⁻¹) | 1.6 (1.4-1.8) | 556 | 1.0 (0.9-1.2) | 590 | <0.001 | 1.5 (1.3-1.8) | 275 | 1.5 (1.2-1.8) | 285 | 0.70 |

1. Method for identifying a compound as a candidate drug, comprising the steps
a. bringing said compound into contact with a cell expressing the genes GYPC, AGPAT3, AGL, PVRL2, HMGB3, HSDL2; and
b. analyzing if said compound modulates the expression of at least one of said genes.

2. Method according to claim 1, wherein step b comprises analysis of modulation of expression of at least two of said genes.

3. Method according to claim 1 wherein step b further comprises analysis of modulation of expression of a gene selected from the group consisting of CD36 and PPARα.

4. Method for identifying a compound as a candidate drug, comprising
a. bringing said compound into contact with a gene product of a gene selected from the group consisting of GYPC, AGPAT3, AGL, PVRL2, HMGB3, HSDL2; and
b. analyzing if said compound modulates the biological activity of said gene product.

5. Method according to claim 4, wherein the analysis is directed to an increase of the biological activity of said gene product.

6. Method according to claim 4, wherein the analysis is directed to a decrease of the biological activity of said gene product.

7. Method according to claim 4, wherein the biological activity is regulation of expression of a gene implicated in development or progression of atherosclerosis or atherosclerosis-related diseases.

8. Method according to claim 4, wherein the biological activity is regulation of a gene selected from the group consisting of GYPC, AGPAT3, AGL, PVRL2, HMGB3, HSDL2, CD36 and PPARα.

9. Method for identifying a compound as a candidate drug, comprising the steps
a. bringing said compound into contact with a cell expressing the gene LDB2; and
b. analyzing if said compound modulates the expression of LDB2.

10. Method for identifying a compound as a candidate drug, comprising the steps
a. bringing said compound into contact with a gene product of the gene LDB2; and
b. analyzing if said compound modulates the biological activity of LDB2.

11. Method according to claim 10, wherein the biological activity is regulation of expression of a gene implicated in development or progression of atherosclerosis or atherosclerosis related diseases.

12. Method according to claim 10, wherein the biological activity is transendothelial migration of leukocytes.

13. Method according to claim 1, comprising
a. obtaining a DNA molecule comprising the coding sequence of a gene selected from the group consisting of LDB2, GYPC, AGPAT3, AGL, PVRL2, HMGB3, HSDL2, and optionally sequence elements regulating the expression of said gene;
b. introducing said DNA molecule in a host cell, such as a cell line or a cell of a non-human embryo, to obtain cellular expression of said DNA molecule,
c. bringing said host cell into contact with said compound, and
d. analyzing if said compound modulates the expression of said DNA molecule or the biological activity of said gene product.

14. Method according to claim 13, wherein the analysis step comprises the analysis of transendothelial migration of leukocytes.

15. Method according to claim 1, for identifying a compound as a candidate drug for the treatment of a disease selected from the group consisting of atherosclerosis, atherosclerosis-related diseases and inflammatory diseases.

16. Method according to claim 1, wherein the compound is selected from the group consisting of small organic molecules, peptides, polypeptides, proteins, antibodies and fragments thereof, nucleic acids such as DNA or RNA, including siRNA and miRNA, modified nucleic acids, such as PNA, or such compounds modified for enhanced therapeutic purposes.

17. Method for identifying a genetic marker for assessing the predisposition for, development and/or outcome of, atherosclerosis, atherosclerosis-related diseases or inflammatory diseases, comprising
a. detecting genetic variations in a gene selected from the group consisting of the genes LDB2, GYPC, AGPAT3, AGL, PVRL2, HMGB3, HSDL2 between individuals in a population, and
b. correlating said genetic variations to differences in predisposition for, development and/or outcome of, atherosclerosis and atherosclerosis-related diseases, between said individuals.

18. Method according to claim 16, wherein said genetic variation is a variation modulating the expression of a gene product.

19. Method according to claim 16, wherein said genetic variation is a variation modulating the biological activity of a gene product.

20. Genetically modified cell of an animal species
a. comprising a heterologous DNA molecule comprising the coding sequence of a gene selected from the group consisting of the genes LDB2, GYPC, AGPAT3, AGL, PVRL2, HMGB3, HSDL2, and/or
b. having a gene selected from the group consisting of the genes LDB2, GYPC, AGPAT3, AGL, PVRL2, HMGB3, HSDL2 inactivated.

21. Genetically modified cell according to claim 20, wherein the DNA-molecule encodes the LIM Domain Binding protein 2 and/or the gene selected from the group is LDB2.

22. Genetically modified cell according to claim 20, wherein the animal species is a mammal.

23. Genetically modified cell according to claim 20, wherein the animal species is selected from the group consisting of human, non-human primates, and rodents.

24. Genetically modified non-human animal, comprising a cell according to claim 20.

25. Method for treatment of a patient suffering from, or being at risk of developing, atherosclerosis or atherosclerosis-related diseases comprising administering to said patient an original or modified variant of a gene selected from the group consisting of the genes LDB2, GYPC, AGPAT3, AGL, PVRL2, HMGB3, HSDL2 or a compound identified with the method according to claim 1.

26. Method according to claim 25, wherein the administered gene is a gene encoding LIM Domain Binding 2.

27. Method according to claim 25, wherein the compound is a siRNA.

28. Method for treatment of a patient suffering from, or being at risk of developing, atherosclerosis or atherosclerosis-related diseases comprising administering to said patient a compound selected from the group consisting of siRNA molecules targeting a gene selected from the group consisting of LDB2, GYPC, AGPAT3, AGL, PVRL2, HMGB3, HSDL2.

29. Method for identifying a subject as having an lower than average risk of developing atherosclerosis or atherosclerosis-related diseases, comprising analyzing the LDB2 gene of said subject and wherein the presence of the T minor allele of the single nucleotide polymorphism rs10939673 in the LDB2 gene indicates a lower than average risk.
